SEQUENCE LISTING (4 sequences)

SEQ ID NO: 1 - DNA, 1491 bp, Prevotella copri
  misc_feature: Prevotella copri gene for 16S ribosomal RNA, partial sequence, strain CB18 GenBank AB244770.1
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaycacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagyctgaaccagccaagtagcgtgcaggawgacggccctatgggttgtaaactgcttttataagggaataaagtgagcctcgtgagrctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctgggggagtacgccggcaacggtgaaactcaaaggaattgacggggcccgcacaagcggaggaacatgtggttaattcgatgatacgcgaggaaccttacccgggcttgaattgcagaggaaggattggagacaatgacgcccttcggggcctctgtgaaggtgctgcatggttgtcgtcagctcgtgccgtgaggtgtcggcttaagtgccataacgagcgcaacccctctccttagttgccatcaggtyawgctgggcactctggggacactgccaccgtaaggtgtgaggaaggtggggatgacgtcaaatcagcayggcccttacgtccggggctacacacgtgttacaatggcaggtacagagagacggtysywygyaarwtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 2 - DNA, 1344 bp, Veillonella rogosae
  misc_feature: Veillonella rogosae strain CF100 16S ribosomal RNA gene, partial sequence GenBank EF108443.1
cgcgtaatcaacctgcccttcagagggggacaacagttggaaacgactgctaataccgcatacgatccaacctcggcatcgaggatggatgaaaggtggcctctatttataagctatcactgaaggaggggattgcgtctgattagctagttggaggggtaacggcccaccaaggcaatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggtcctcttgcgaatagttagaggaattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacgtaatggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtgg

SEQ ID NO: 3 - DNA, 1487 bp, Odoribacter laneus
  misc_feature: Odoribacter laneus gene for 16S ribosomal RNA, partial sequence, strain JCM 16069 GenBank AB547648.1
agagtttgatcctggctcaggatgaacgctagcgacaggcttaacacatgcaagtcgaggggtaacagggtgtagcaatacaccgctgacgaccggcgcacgggtgagtaacgcgtatgcaacctgcctttgacagagggatagcccatggaaacgtggattaatacctcatagtctctttttccttcctggggaatagagtaaaacgagagtggtcaaagatgggcatgcgtcctattaggcagttggcggggtaacggcccaccaaaccgatgataggtaggggttctgagaggaaggtcccccacactggtactgagacacggaccagactcctacgggaggcagcagtgaggaatattggtcaatggtcgagagactgaaccagccaagtcgcgtgagggatgactgccctatgggttgtaaacctcttttctactgggagaataagccttatgtatagggtgatgacagtacagtaggaataagcatcggctaactccgtgccagcagccgcggtaatacggaggatgcgagcgttatccggatttattgggtttaaagggtgcgtaggcggctttataagttagtggtaaaatttcggagcttcactccggtccgccattaaaactgtagagctagagaatggacgaggtaggcggaataagttaagtagcggtgaaatgcatagatataacttagaactccgatagcgaaggcagcttaccagaccataactgacgctgatgcacgagagcgtgggtagcgaacaggattagataccctggtagtccacgccgtaaacgatgctcaccggcccttagcgataagacagttaggggttaattgaaagaattaagtgagccacctggggagtacgtcggcaacgatgaaactcaaaggaattgacgggggcccgcacaagcggaggaacatgtggtttaattcgatgatacgcgaggaaccttacctgggtttaaatgtatattgcataatctggaaacagtttttctcttcggagctatatacaaggtgctgcatggttgtcgtcagctcgtgccgtgaggtgtcgggttaagtcccataacgagcgcaacccttaccgttagttgctaacatgtaatgatgagcactctagcgggactgccaccgtaaggtgagaggaagggggggatgacgtcaaatcagcacggcccttacatccagggcgacacacgtgttacaatggccataacagcgggtagctaccgggtgaccggatgcaaatctcgaaaattggtctaagttcggattggagtctgcaacccgactccatgaagttggattcgctagtaatcgcgcatcagccatggcgcggtgaatacgttcccgggccttgtacacaccgcccgtcaagccatggaagctgggagtacctgaagtccgtaaccgcgaggatcggcctagggtaataccggtaactggggctaagtcgtaacaaggtaacc

SEQ ID NO: 4 - DNA, 1488 bp, Clostridium ramosum
  misc_feature: Clostridium ramosum gene for 16S ribosomal RNA, partial sequence, strain JCM 5234 GenBank AB627078.1
agagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaaggaaggagctgtctaaggtgggattgatgattggggtgaagtcgtaacaaggtaacc

(19) United States
(12) Patent Application Publication
(10) Pub. No.: US 2020/0347440 A1
(43) Pub. Date: Nov. 5, 2020

(54) TARGETED INTERVENTIONS DIRECTED AT REDUCING THE LEVELS OF CIRCULATING SUCCINATE IN A SUBJECT, AND KITS AND METHOD FOR DETERMINING EFFECTIVENESS OF SAID INTERVENTIONS

(71) Applicants: CONSORCIO CENTRO DE INVESTIGACIÓN BIOMÉDICA EN RED, M.P., Madrid (ES); INSTITUT D'INVESTIGACIÓ SANITÀRIA PERE VIRGILI, Tarragona (ES); UNIVERSITAT ROVIRA I VIRGILI, Tarragona (ES)

(72) Inventors: Sonia FERNÁNDEZ VELEDO, Tarragona (ES); Carolina SERENA PERELLÓ, Tarragona (ES); Joan Josep VENDRELL ORTEGA, Tarragona (ES); Victoria CEPERUELO MALLAFRÉ, Tarragona (ES); Enrique CALVO MANSO, Tarragona (ES)

(21) Appl. No.: 16/962,867

(22) PCT Filed: Jan. 17, 2019
(86) PCT No.: PCT/EP2019/051157

§ 371 Date: Jul. 17, 2020

(30) Foreign Application Priority Data
Jan. 17, 2018 (EP) 18382020.8

(51) Int. Cl. C12Q 1/689; A61K 35/741

(52) U.S. Cl. CPC C12Q 1/689; A61K 35/741

(57) ABSTRACT

The present invention relates to kits suitable for determining the ratio of succinate-producing bacteria to succinate-consuming bacteria in a stool sample from a subject, in particular the (Prevotellaceae+Veillonellaceae)/(Odoribacteriaceae+Clostridiaceae) ratio, or for determining the succinate level in a biofluid sample from a subject. The invention also relates to a method for determining whether a targeted intervention directed at reducing the levels of circulating succinate in a subject has been effective. Finally, the invention relates to targeted interventions for use in the prevention and/or treatment of a disease associated with increased levels of circulating succinate in a patient.

### FIELD OF THE INVENTION

The present invention relates to targeted interventions directed at reducing the levels of circulating succinate in a subject. The invention further relates to kits and to a method for determining effectiveness of said interventions.

### BACKGROUND

Cardiovascular disease (CVD) is a collective term used to describe heart and blood vessel disorders and constitutes the leading cause of death worldwide. In developed countries, CVD usually manifests as coronary artery disease, atherosclerosis and hypertension, with central obesity playing an increasingly important role as a risk factor.

The generation of reactive oxygen species and consequent downstream ramifications has been associated to the progression of CVD. Elevated levels of circulating succinate have also been detected in several high-risk CVD states such as hypertension (Sadagopan et al., 2007, Am. J. Hypertens. 20:1209-1215), ischemic heart disease (Aguiar et al., 2014, Cell Commun. Signal. 12:78) and type 2 diabetes mellitus (T2DM) (Guo et al., 2017, Nat. Commun. 8:15621; Sadagopan et al., 2007, Am. J. Hypertens. 20:1209-1215; Toma et al., 2008, J. Clin. Invest. 118:2526-2534; van Diepen et al., 2017, Diabetologia 60:1304-1313). Under these scenarios, extracellular succinate is thought to signal through its cognate receptor SUCNR1/GPR91, with pathological implications in hypertrophic cardiomyopathy (Aguiar et al., 2014, Cell Commun. Signal. 12:78), obesity-related metabolic disturbances (McCreath et al., Diabetes. 2015 April; 64(4):1154-67), renin-induced hypertension (Toma et al., 2008, J. Clin. Invest. 118:2526-2534), and diabetic retinopathy (Ariza et al., 2012, Front. Endocrinol. (Lausanne) 3: 22).

Thus, considering said downstream effects, the reduction of the levels of circulating succinate appears as an attractive strategy for the treatment of different diseases, including CVD and CVD-related pathologies. The succinate receptor has also been suggested as a promising drug target to counteract or prevent cardiovascular and fibrotic defects (Ariza et al., 2012, Front. Endocrinol. (Lausanne) 3:22). Interestingly, the exact origin of circulating succinate is still unclear. On this regard, it has been suggested that damaged tissues may contribute to the succinate found in circulation (Ariza et al., 2012, Front. Endocrinol. (Lausanne) 3: 22; Deen and Robben, 2011, J. Am. Soc. Nephrol. 22:1416-1422). Accordingly, there is a need in the art for efficient interventions directed at reducing the levels of circulating succinate in a subject.

### BRIEF SUMMARY OF THE INVENTION

The inventors have surprisingly found that succinate produced by bacteria of the gut microbiota is an essential contributor to total circulating succinate levels. Further, they have been able to demonstrate that the ratio of succinate-producing bacteria to succinate-consuming bacteria, in particular the (Prevotellaceae+Veillonellaceae)/(Odoribacteriaceae+Clostridiaceae) ratio, measured in a stool sample from a subject can be related to circulating succinate levels in the same subject.

Thus, in a first aspect the invention relates to a kit comprising reagents suitable for determining the ratio of succinate-producing bacteria to succinate-consuming bacteria in a stool sample from a subject,
wherein the kit comprises primer sets designed to specifically hybridize the hypervariable regions of the 16S rRNA gene in at least one succinate-producing bacterium and in at least one succinate-consuming bacterium, or
wherein the kit comprises probes that specifically hybridize to the hypervariable regions of the 16S rRNA gene in at least one succinate-producing bacterium and in at least one succinate-consuming bacterium,

and wherein the primer sets or the probes comprise at least 10% of the total amount of reagents forming the kit.

In a second aspect, the invention relates to the use of the kit according to the first aspect of the invention to detect the ratio of succinate-producing bacteria to succinate-consuming bacteria in a stool sample from a subject.

In a third aspect, the invention relates to a kit comprising reagents suitable for determining the succinate level in a biofluid sample from a subject,
wherein the presence of succinate in said biofluid sample above a predetermined threshold level provides a positive result and
wherein the presence of succinate in said biofluid sample below a predetermined threshold level or the absence of succinate in said biofluid sample provides a negative result.

In a fourth aspect, the invention relates to the use of the kit according to the third aspect of the invention to determine whether the succinate level in a biofluid sample from a subject is above a threshold level.

In another aspect, the invention relates to the use of a kit to determine whether a probiotic intervention directed at reducing the levels of circulating succinate in a subject has been effective, the kit comprising reagents suitable for determining the succinate level in a biofluid sample from a subject, wherein
a level of circulating succinate in the biofluid sample from the subject after the probiotic intervention lower than the level of circulating succinate in the biofluid sample from the subject before the probiotic intervention is indicative that the probiotic intervention has been effective,
   and wherein
a level of circulating succinate in the biofluid sample from the subject after the probiotic intervention equal to or higher than the level of circulating succinate in the biofluid sample from the subject before the probiotic intervention is indicative that the probiotic intervention has not been effective.

In a further aspect, the invention relates to a method for determining whether a targeted intervention directed at reducing the levels of circulating succinate in a subject has been effective, the method comprising:
(a) determining the ratio of succinate-producing bacteria to succinate-consuming bacteria in a stool sample from the subject before the targeted intervention, and
(b) determining the ratio of succinate-producing bacteria to succinate-consuming bacteria in a stool sample from the subject after the targeted intervention,

wherein
a ratio of succinate-producing bacteria to succinate-consuming bacteria in the stool sample from the subject after the targeted intervention lower than the ratio of succinate-producing bacteria to succinate-consuming bacteria in the stool sample from the subject before the targeted intervention is indicative that the targeted intervention has been effective,

and wherein
a ratio of succinate-producing bacteria to succinate-consuming bacteria in the stool sample from the subject after the targeted intervention equal to or higher than the ratio of succinate-producing bacteria to succinate-consuming bacteria in the stool sample from the subject before the targeted intervention is indicative that the targeted intervention has not been effective.

In yet a further aspect, the invention relates to a dietary intervention or diet product for use in the prevention and/or treatment of a disease associated with increased levels of circulating succinate in a patient, wherein the intervention decreases the ratio of succinate-producing bacteria to succinate-consuming bacteria in the intestinal tract of the patient.

In still a further aspect, the invention relates to a product for use in the prevention and/or treatment of a disease associated with increased levels of circulating succinate in a patient, wherein the product decreases the ratio of succinate-producing bacteria to succinate-consuming bacteria in the intestinal tract of the patient, wherein the product is selected from the group consisting of a pharmacological product, and a probiotic product.

In another further aspect, the invention relates to a product for use in the prevention and/or treatment of a disease associated with increased levels of circulating succinate in a patient, wherein the product decreases the levels of circulating succinate of the patient, wherein the product is selected from the group consisting of a pharmacological product, and a probiotic product.

In a final aspect, the invention relates to a probiotic product comprising an effective amount of succinate consuming bacteria, wherein the succinate consuming bacteria is selected from the group consisting of Odoribacter spp, Phascolarctobacterium spp, Ruminococcus spp and combinations thereof.

### BRIEF DESCRIPTION OF THE FIGURES

FIG. 1 shows the decision tree for identification of predictors of optimal/altered metabolic profile. Classification and regression tree for optimal/altered metabolic profile based on age, body mass index (BMI), succinate, cholesterol, High-density lipoprotein-c (HDLc), Systolic blood pressure (SBP), Diastolic blood pressure (DBP) and type 2 diabetes (T2DM). Pie charts represent the proportion of patients who met optimal (dark gray) or altered (light gray) at each node of the tree.

FIG. 2 shows that circulating succinate levels are increased in obesity and type 2 diabetes. (A) Circulating plasma levels in lean, obese and type 2 diabetes (T2DM) individuals. Data are expressed as median and interquartile range. Differences were analyzed by the Kruskal-Wallis test with post hoc Dunn's multiple comparison test. *, p<0.0001 vs. lean. (B) Positive correlation between succinate levels and BMI, insulin, glucose, HOMA-IR and triglycerides using the entire cohort. (C) Negative correlation between succinate levels and levels of SAT ATGL, SAT ABHD5, SAT HSL and SAT ZAG. (D) Positive correlation between succinate levels and SAT HIF1A and SAT CD163. SAT; subcutaneous adipose tissue. Statistical analyses for (B), (C) and (D): Spearman's correlation analysis.

FIG. 3 shows that obese-gut microbiota composition is associated with circulating succinate levels. (A) Percentage of incidence within Bacteroidetes and Firmicutes families in non-obese and obese individuals. (B) Differences between non-obese and obese individuals at the family level: families [(Prevotellaceae plus Veillonellaceae)/(Odoribacteriaceae plus Clostridaceae)] (fam[(P+V)/(O+C)]) ratio. (C) Positive correlation between succinate serum levels and fam[(P+V)/(O+C)]) ratio. (D) Positive correlation between succinate serum levels and circulating zonulin levels. (E) Validation studies were performed using cohort III. Percentage of incidence within Bacteroidetes and Firmicutes families in lean and obese individuals. (F) Positive correlation between succinate plasma levels and Veillonellaceae. (G) Differences between lean and obese individuals in the fam[(P+V)/(O+C)] ratio in the cohort III study. (H) Positive correlation between succinate serum levels and log fam[(P+V)/(O+C)]) ratio in the cohort III study. Data information: For (A) and (E), values are expressed as mean SD. For (B) and (G), data are represented in box and whisker plot format (whiskers: min to max). Statistical analyses: Mann-Whitney U-test. *, p<0.05 vs. non-obese or lean. For (C), (D), (F) and (H) Spearman's or Pearson's correlation analysis with Bonferroni adjustment were used. (I) Seventeen type 2 diabetes mellitus (T2D) subjects (9 women and 4 men) were included in the study. The P+V/O+C ratio is 4.70±6.12. (J) Graph shows Spearman correlation between succinate plasma levels and Odoribacteraceae for 26 plasma samples from obese diabetic patients. Subjects were recruited at the Endocrinology Service at the Hospital Universitari de Bellvitge (Barcelona, Spain).

FIG. 4 shows the weight loss induced by dietary intervention or diet product modifies specific-gut microbiota and impacts circulating succinate levels. (A) Circulating serum succinate levels in basal state and after a 12-week dietary intervention or diet product (12-wDI) from cohort IV. (B) Percentage of incidence within Bacteroidetes and Firmicutes families in obese individuals in basal state and after 12-wDI. (C) Positive correlation between the change in succinate serum levels (12-wDI [succinate]−basal [succinate]) and the change in Prevotellaceae (12-wDI [% abundance Prevotellaceae]-basal [% abundance Prevotellaceae]). (D) Differences between basal state and 12-wDI in the fam[(P+V)/(O+C)] ratio. (E) Positive correlation between the change in succinate serum levels (12-wDI [succinate]−basal [succinate]) and the change in the (12-wDI fam[P+V/O+C]−basal fam[(P+V)/(O+C)]) ratio. Data information: For (A) and (B) values are expressed as mean SD. For (D) data are represented in box and whisker plot format (whiskers: min to max). Statistical analyses: Wilcoxon signed-rank test. *, p<0.05 vs. basal. For (C) and (D), Spearman's correlation analysis with Bonferroni adjustment was used.

FIG. 5 shows the Gut microbiota composition in non-obese and obese subjects in the cohort II study. (A) Firmicutes/bacteroidetes ratio (B) richness index (number of OTUs) and (C) diversity index (Shannon-Weaver) calculated in non-obese and obese individuals. (D) Percentage of incidence within Bacteroidetes and Firmicutes genera in non-obese and obese individuals. (E) Ratio at genus level [(Prevotella spp. Plus Veillonella spp.)/(Odoribacter spp. plus Clostridium spp.)] (gen[(P+V)/(O+C)]) in non-obese and obese individuals. Data information: For (A), (B), (C) and (E) data are represented in box and whisker plot format (whiskers: min to max). For (D) values are expressed as mean value SD. Statistical analyses: U de Mann-Whitney test. *, p<0.05 vs. non-obese.

FIG. 6 shows the Gut microbiota composition in the dietetic intervention study cohort IV. (A) Richness index (number of OTUs) and (B) diversity index (Shannon-Weaver) (C) Firmicutes/bacteroidetes ratio calculated in basal state and at 12-wDI in obese individuals from the microbiota cohort IV. (D) Differences in percentage of incidence within Bacteroidetes and Firmicutes genera in basal state and at 12-wDI. (E) Ratio at genus level (gen[(P+V)/(O+C)]) in basal state and at 12-wDI. Data information: For (A), (B), (C) and (E) data are represented in box and whisker plot format (whiskers: min to max). For (D) values are expressed as mean value SD. Statistical analyses: Wilcoxon signed-rank test. *, p<0.05 vs 12-wDI.

FIG. 7 shows metabolic genes related to succinate metabolism and succinate metabolizing microbiota. Differences in genes encoding enzymes between group 1 (patients ratio decreases at the end of follow-up) and group 2 (patients ratio increases at the end of follow-up). Data are represented in scatter dot plot with mean and SD. Statistical analyses: U de Mann-Whitney test.

FIG. 8 shows the effect of Odoribacter laneus on Glucose Tolerance Test (GTT) in obese mice. C57/B16 mice were fed with a High Fructose Diet for 16 weeks. Resultant obese mice were then daily treated with 100 uL Odoribacter laneus at 1×10⁹ CFU/mL in PBS+glycerol 1% (vehicle) with an oral gavage for 24 days. Glucose Tolerance Test (A) improved in Odoribacter laneus-treated animals. The area under the curve (AUC) is shown in (B).

### DETAILED DESCRIPTION OF THE INVENTION

As explained above, the inventors have surprisingly found that succinate produced by bacteria of the gut microbiota is an essential contributor to total circulating succinate levels. Further, they have been able to demonstrate that the ratio of succinate-producing bacteria to succinate-consuming bacteria, in particular the (Prevotellaceae+Veillonellaceae)/(Odoribacteriaceae+Clostridiaceae) ratio, measured in a stool sample from a subject can be related to circulating succinate levels in the same subject.

Kits of the Invention

The inventors have developed kits for determining the ratio of succinate-producing bacteria to succinate-consuming bacteria in the intestinal tract of the patient.

Thus, in a first aspect, the invention relates to a kit comprising reagents suitable for determining the ratio of succinate-producing bacteria to succinate-consuming bacteria in a stool sample from a subject, preferably
wherein the kit comprises primer sets designed to specifically hybridize the hypervariable regions of the 16S rRNA gene in at least one succinate-producing bacterium and in at least one succinate-consuming bacterium, or
wherein the kit comprises probes that specifically hybridize to the hypervariable regions of the 16S rRNA gene in at least one succinate-producing bacterium and in at least one succinate-consuming bacterium,

and wherein the primer sets or the probes comprise at least 10% of the total amount of reagents forming the kit.

In the context of the present invention, “kit” is understood as a product containing the different reagents for use in accordance with the different uses and methods of the invention packed so as to allow their transport and storage. Additionally, the kits used in the invention can contain instructions for the simultaneous, sequential or separate use of the different components which are in the kit. Said instructions can be in the form of printed material or in the form of an electronic support capable of storing instructions susceptible of being read or understood, such as, for example, electronic storage media (e.g. magnetic disks, tapes), or optical media (e.g. CD-ROM, DVD), or audio materials. Additionally or alternatively, the media can contain internet addresses that provide said instruction.

In a preferred embodiment, the kit comprises primer sets designed to specifically hybridize the hypervariable regions of the 16S rRNA gene in at least one succinate-producing bacterium and in at least one succinate-consuming bacterium.

In another preferred embodiment, the kit comprises probes that specifically hybridize to the hypervariable regions of the 16S rRNA gene in at least one succinate-producing bacterium and in at least one succinate-consuming bacterium.

The term “16S rRNA gene” as used herein, refers to a bacterial gene encoding the component of the 30S small subunit of a prokaryotic ribosome that binds to the Shine-Dalgarno sequence. Sequence analysis of the 16S ribosomal RNA (rRNA) gene has been widely used to identify bacterial species and perform taxonomic studies. Bacterial 16S rRNA genes generally contain nine “hypervariable regions” that demonstrate considerable sequence diversity among different bacterial species and can be used for species identification. Thus, the term “hypervariable regions of the 16S rRNA gene”, as used herein, refers to said sequences in the 16S ribosomal rRNA gene, that allow identifying a single bacterial species or differentiating among a limited number of different species or genera. In the context of the present invention, the hypervariable regions of the 16S rRNA gene allow to identify or differentiate at least one succinate-producing bacterium and at least one succinate-consuming bacterium. The identification of said regions can be mediated by techniques well known by the person skilled in the art. Non-limiting examples of such techniques are polymerase chain reaction (PCR) amplification, Real Time polymerase chain reaction (RT-PCR), In situ Hybridization (ISH), Northern blot or Micro-array.

In a particular embodiment, the hypervariable regions of the 16S rRNA gene are used to identify bacteria of the species Prevotella spp., Veillonella spp., Odoribacter spp. and/or Clostridium spp. In a preferred embodiment, the Prevotella spp. gene for 16S rRNA comprises a sequence that has at least 85%, at least 90%, at least 95%, at least 99% or at least 100% identity with SEQ ID No: 1 (Genbank access No.: AB244770; version No.: AB244770.1; date of last modification 19 Apr. 2007). In a more preferred embodiment, the Prevotella spp. is Prevotella copri and the gene for 16S rRNA comprises the sequence with SEQ ID No: 1. In a preferred embodiment, the Veillonella spp. gene for 16S rRNA comprises a sequence that has at least 90%, at least 95%, at least 99% or at least 100% identity with SEQ ID No: 2 (Genbank access No.: EF108443; version No.: EF108443.1, date of last modification 3 Jan. 2011). In a more preferred embodiment, the Veillonella spp. is Veillonella rogosae and the gene for 16S rRNA comprises the sequence with SEQ ID No: 2. In a preferred embodiment, the Odoribacter spp. gene for 16S rRNA comprises a sequence that has at least 86%, at least 90%, at least 95%, at least 99% or at least 100% identity with SEQ ID No: 3 (Genbank access No.: AB547648; version No.: AB547648.1; date of last modification 9 Nov. 2012). In a more preferred embodiment, the Odoribacter spp. is Odoribacter laneus and the gene for 16S rRNA comprises the sequence with SEQ ID No: 3. In a preferred embodiment, the Clostridium spp. gene for 16S rRNA comprises a sequence that has at least 95%, at least 99% or at least 100% identity with SEQ ID No: 4. In a more preferred embodiment, the Clostridium spp. is Clostridium ramosum and the gene for 16S rRNA comprises the sequence with SEQ ID No: 4 (Genbank access No.: AB627078; version No.: AB627078.1, date of last modification 9 Nov. 2012).

The term “primer set”, as used herein, refers to a set of oligonucleotides of RNA or DNA (preferably of about 15-35 bases) that specifically hybridizes to the hypervariable regions of the 16S rRNA gene and serves as a starting point for DNA synthesis. They are required for DNA amplification mediated by a DNA polymerase in a reaction based on the PCR technique. The relative amount, concentration and/or average size of each amplicon can then be analyzed with techniques known by the person skilled in the art. Non-limiting examples of such techniques are gel electrophoresis, or techniques based on the RT-PCR technique. It is also possible to sequence the target nucleic acid using said primers and after further steps known to the expert in the field.

The term “probe”, as used herein, refers to DNA or RNA oligonucleotide sequences that hybridize by complementarity with a specific sequence. In other words, the probe hybridizes to specific single-stranded nucleic acid (DNA or RNA) whose base sequence allows probe-target base pairing due to complementarity between the probe and the target. In a preferred embodiment, the subsequent hybrid can be detected using techniques known by the expert in the field. For instance, the probe can be labelled with a marker that can be radioactive or (a) fluorescent molecule(s) and immobilized on a membrane or in situ. Commonly used markers are 32P (a radioactive isotope of phosphorus incorporated into the phosphodiester bond in the probe DNA) or Digoxigenin, which is a non-radioactive, antibody-based marker. DNA sequences or RNA transcripts that have moderate to high sequence similarity to the probe are then detected by visualizing the hybridized probe via autoradiography or other imaging techniques. Normally, either X-ray pictures are taken of the filter, or the filter is placed under UV light, or under a microscope for the detection of the fluorescently labelled probe. Detection of sequences with moderate or high similarity depends on how stringent the hybridization conditions were applied-high stringency, such as high hybridization temperature and low salt in hybridization buffers, permits only hybridization between nucleic acid sequences that are highly similar, whereas low stringency, such as lower temperature and high salt, allows hybridization when the sequences are less similar.

The term “oligonucleotide”, as used herein, refers to a single-stranded DNA or RNA molecule, preferably with up to 35, 30, 25, 20, 19, 18, 17, 16, 15, 14 or 13 bases in length (upper limit). The oligonucleotides of the invention are DNA or RNA molecules, preferably of at least 2, at least 5, at least 10, at least 12, at least 15, at least 16, at least 17, at least 18, at least 19, at least 20, at least 21, at least 22, at least 23, at least 25 nucleotide bases in length (lower limit). Ranges of base lengths can be combined in all different manners using the afore-mentioned lower and upper limits, for example at least 2 and up to 30 bases, at least 10 and up to 15 bases, at least 5 and up 15 bases or at least 15 and up to 18 bases.

The term “specifically hybridize”, as used herein, refers to the conditions which allow the hybridization of two polynucleotides under high stringent conditions or moderately stringent conditions. The “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and the target sequence, the higher the relative temperature which must be used. As a result, it follows that higher relative temperatures would tend to make the reaction conditions more stringent, while lower temperatures less so. See Brown T, “Gene Cloning” (Chapman & Hall, London, U K, 1995).

In preferred embodiments, the primer sets or the probes comprise at least 10%, at least 20%, at least 30%, at least 40%, at least 50%, at least 60%, at least 70%, at least 80%, at least 90% or at least 100% of the total amount of reagents forming the kits of the invention. In a particular embodiment, the total amount of reagents forming the kits of the invention refers to the total number of reagents in the kit.

The term “succinate”, as used herein, refers to a metabolite that is the anion of succinic acid, and is also known as butanedioate. It is an intermediate of the tricarboxylic acid (TCA) cycle, and plays a crucial role in adenosine triphosphate (ATP) generation in mitochondria. The chemical formula of succinate is C₄H₄O₄²⁻. The expression “circulating succinate”, or “circulating succinate in a subject”, as used herein, refers to succinate detectable in a blood, plasma or serum sample from a subject.

The expression “succinate-producing bacteria”, as used herein, refers to bacteria that produce and release succinate. In the context of the present invention, the expression “succinate-producing bacteria” refers equally to bacteria which are actively producing succinate and to bacteria which are not actively producing succinate, provided that the latter are capable of producing and releasing succinate wherein the environmental conditions so permit (i.e. in the presence of the appropriate substrates). In a particular embodiment, the “succinate-producing bacteria” is a bacteria which is actively producing succinate. In another particular embodiment, the “succinate-producing bacteria” is a bacteria which is not actively producing succinate, provided that it is capable of producing and releasing succinate wherein the environmental conditions so permit (i.e. in the presence of the appropriate substrates). A person skilled in the art would be able to design an assay in order to determine whether a bacterium is a succinate-producing bacterium. In example, the bacterium may be growth in culture medium for a predetermined length of time and the conditioned culture medium may then be analyzed for the accumulation of succinate in said culture medium, which is indicative that the bacterium is a succinate-producing bacterium. Examples of succinate producing bacteria are known in the art: Louis et al., 2014, Nat. Rev. Microbiol 12:661-672; Nakayama et al., 2017, Front. Microbiol. 8:197; Vogt et al., 2015, Anaerobe 34:106-115. Preferably, said bacteria are intestinal bacteria, i.e., that can survive and multiply efficiently in the intestine of a subject. In a preferred embodiment said bacteria are selected from the group consisting of Prevotella spp., Veillonella spp., Bacteroides spp. Paraprevotella spp., Succinovibrio spp., Ruminococcus spp., Fibrobacter succinogenes and combinations thereof. More preferably the succinate-producing bacteria are those from the family Prevotellaceae and Veillonellaceae. The family Prevotellaceae belongs to the phylum Bacteroidetes, the class Bacteroidia, and the order Bacteroidales. It is composed of four genera: Prevotella, Alloprevotella, Hallella, and Paraprevotella. The family Veillonellaceae belongs to the phylum Firmicutes, the class Negativicutes, and the order Selenomonadales. It includes 6 genera: Veillonella, Megasphaera, Dialister, Allisonella, Anaeroglobus, and Negativicoccus. Still more preferably, the succinate-producing bacteria are a species selected from the group consisting of Prevotella copri; Prevotella intermedia; Prevotella nigrescens; Prevotella melaninogenica; Prevotella nanceiensis; Veillonella rogosae; Veillonella atypical and combinations thereof. Prevotella and Veillonela are both gram negative bacteria.

The expression “succinate-consuming bacteria”, as used herein, refers to bacteria that consume succinate. In the context of the present invention, the expression “succinate-consuming bacteria” refers equally to bacteria which are actively consuming succinate and to bacteria which are not actively consuming succinate, provided that the latter are capable of consuming succinate wherein the environmental conditions so permit (i.e. in the presence of succinate). In a particular embodiment, the “succinate-consuming bacteria” is a bacteria which is actively consuming succinate. In another particular embodiment, the “succinate-consuming bacteria” is a bacteria which is not actively consuming succinate, provided that it is capable of consuming succinate wherein the environmental conditions so permit (i.e. in the presence of succinate). A person skilled in the art would be able to design an assay in order to determine whether a bacterium is a succinate-consuming bacterium. In example, the bacterium may be growth in culture medium containing succinate for a predetermined length of time and the conditioned culture medium may then be analysed for the depletion of succinate in said culture medium and the accumulation of an end product such as butyrate, which is indicative that the bacterium is a succinate-consuming bacterium. Examples of succinate consuming bacteria are known in the art: Ferreyra et al., 2014, Cell Host Microbe. 16:770-777; Reichardt et al., 2014, ISME J. 8:1323-1335. Preferably, said bacteria are intestinal bacteria, i.e., that can survive and multiply efficiently in the intestine of a subject. In a preferred embodiment said bacteria are selected from the group consisting of Odoribacter spp., Clostridium spp, Phascolarctobacterium succinatutens and combinations thereof. More preferably the succinate-consuming bacteria are those from the family Odoribacteriaceae and Clostridiaceae. The family Odoribacteriaceae belongs to the phylum Bacteroidetes, the class Bacteroidia, and the order Bacteroidales. The family Clostridiaceae belongs to the phylum Firmicutes, the class Clostridia, and the order Clostridiales. Still more preferably, the succinate-producing bacteria are a species selected from the group consisting of Odoribacter laneus; Odoribacter splanchnicus; Clostridium scindens; Clostridium symbiosum; Clostridium perfringens; Clostridium citroniae; Clostridium hathewayi; Clostridium ramosum, and combinations thereof. Odoribacter are gram negative bacteria, whereas Clostridium are gram positive bacteria.

The expression “ratio of succinate-producing bacteria to succinate-consuming bacteria”, as used herein, refers to the result of dividing the total numbers of or a specific subset of succinate-producing bacteria by the total numbers of or a specific subset of succinate-consuming bacteria. In a preferred embodiment, the ratio of succinate-producing bacteria to succinate-consuming bacteria to be determined is the (Prevotellaceae+Veillonellaceae)/(Odoribacteriaceae+Clostridiaceae) ratio.

The expression “stool”, as used herein, refers to the solid or semisolid fecal remains of food that could not be digested in the intestine. In a particular embodiment, a stool sample is collected from a subject after defecation. The term “subject” or “patient”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human of any age or race.

In a second aspect, the invention relates to the use of the kit according to the first aspect of the invention to detect the ratio of succinate-producing bacteria to succinate-consuming bacteria in a stool sample from a subject, preferably the (Prevotellaceae+Veillonellaceae)/(Odoribacteriaceae+Clostridiaceae) ratio.

In a third aspect, the invention relates to a kit comprising reagents suitable for determining the succinate level in a biofluid sample from a subject,
wherein the presence of succinate in said biofluid sample above a predetermined threshold level provides a positive result and
wherein the presence of succinate in said biofluid sample below a predetermined threshold level or the absence of succinate in said biofluid sample provides a negative result.

The expression “reagents suitable for determining the succinate level in a biofluid”, as used herein, refers to reagents that can detect directly or indirectly the presence of succinate in a sample. Non-limiting examples are reagents that can detect the presence of NADPH or Pi, which can be generated in presence of succinate. A non-limiting example of reactions wherein the presence of succinate results in the generation of Pi is the following: Succinate+ATP+CoA converted by succinyl-CoA synthase to succinyl-CoA+ADP+Pi. In a particular example, the color intensity at 450 nm of the reaction product is directly proportional to succinate concentration in the sample. In a preferred embodiment, at least one of the reaction products is detectable by a color change. In another preferred embodiment, the kit comprises a succinate specific enzyme.

In preferred embodiments, the reagents suitable for determining the succinate level in a biofluid comprise each one at least 10%, at least 20%, at least 30%, at least 40%, at least 50%, at least 60%, at least 70%, at least 80%, at least 90% or at least 100% of the total amount of reagents forming the kits of the invention. In a particular embodiment, the total amount of reagents forming the kits of the invention refers to the total number of reagents in the kit.

The term “biofluid”, or “biofluid from a subject”, as used herein, refers to biological fluid obtained from the organism of a subject. Non-limiting examples of biofluid are blood, saliva, cerebrospinal fluid, urine, stool, bone marrow, a nipple aspirate, plasma, serum, cerebrospinal liquid (CSF), feces, a buccal or buccal-pharyngeal swab, a surgical biofluid specimen, or a specimen obtained from a biofluid biopsy. Thus, the expression “biofluid sample”, as used herein, refers to a sample isolated from a biofluid of a subject. Methods for isolating biofluid samples are well known to those skilled in the art.

In a particular embodiment, the biofluid is urine, blood or saliva; preferably urine. In a preferred embodiment wherein the biofluid is urine, the succinate threshold level is preferably between 5 and 15 μM, more preferably between 8 and 12 μM, and still more preferably 10 μM. In a preferred embodiment wherein the biofluid is blood, the succinate threshold level is preferably between 50 and 70 μM, more preferably between 55 and 65 μM, and still more preferably 60 μM.

The expression “threshold level”, as used herein, refers to the concentration level of at least one specific analyte, indicative that a subject is classified as having an abnormal metabolic profile associated with an increased risk of developing metabolic pathologies such as diabetes and therefore is likely to suffer said metabolic pathologies if the analyte level of the patient is above said threshold level. Typically, threshold levels are calculated using the CART (Classification and Regression Tree) statistical method to determine the succinate values characteristic of subjects with an “altered” or subjects with an “optimal” metabolic profile. The main elements of CART are: (a) rules for splitting data at a node based on the value of one variable; (b) stopping rules for deciding when a branch is terminal and can be split no more; and (c) finally, a prediction for the target variable in each terminal node.

In a particular embodiment, the kit is a home test kit. As used herein the term “home test kit” refers to a test kit that involves a subject being able to do the test at home, for example, a urine test which indicates a positive or negative result by a color change or other means such as a digital output. The home test is designed to be used by someone with no medical experience and as such the urine type tests are ideal. The home test kits are sensitive to the presence of succinate in a sample and change color, or otherwise indicate, when above the threshold sensitivity to the succinate is detected in the particular test.

In a particular embodiment, the kit comprises at least one test strip. As used herein a “test strip” is a strip of the kind used for the purpose of placing a sample on a particular spot which initiates a succinate sample color or other indicator test. In newer type testing the test strip could also be a digital type where an indicator screen displays a message such as high succinate present or not present instead of a simple color change. While “strip” in one embodiment means a single device, for purposes of this invention, other embodiments covered by the term test strip includes two or more devices attached to one another for ease in putting urine on both at the same time. In yet another embodiment it refers to two or more separate strips designed to be used at the same time to get the results of the more and less sensitive tests at the same time.

In a fourth aspect, the invention relates to the use of the kit according to the third aspect of the invention to determine whether the succinate level in a biofluid sample from a subject is above a threshold level. In a particular embodiment, the biofluid sample from the subject is a blood sample, a urine sample or a stool sample. The succinate threshold levels are as defined herein above.

In another aspect, the invention relates to the use of a kit to determine whether a probiotic intervention directed at reducing the levels of circulating succinate in a subject has been effective, the kit comprising reagents suitable for determining the succinate level in a biofluid sample from a subject, wherein
a level of circulating succinate in the biofluid sample from the subject after the probiotic intervention lower than the level of circulating succinate in the biofluid sample from the subject before the probiotic intervention is indicative that the probiotic intervention has been effective,

and wherein
a level of circulating succinate in the biofluid sample from the subject after the probiotic intervention equal to or higher than the level of circulating succinate in the biofluid sample from the subject before the probiotic intervention is indicative that the probiotic intervention has not been effective.

Method for Determining Whether a Targeted Intervention has been Effective

The inventors have shown that an intervention that decreases the ratio of succinate-producing bacteria to succinate-consuming bacteria in the intestinal tract of a subject can be effective in reducing the levels of circulating succinate in a subject.

Thus, in a further aspect, the invention relates to a method for determining whether a targeted intervention directed at reducing the levels of circulating succinate in a subject has been effective, the method comprising:
(a) determining the ratio of succinate-producing bacteria to succinate-consuming bacteria in a stool sample from the subject before the targeted intervention, and
(b) determining the ratio of succinate-producing bacteria to succinate-consuming bacteria in a stool sample from the subject after the targeted intervention,

wherein
a ratio of succinate-producing bacteria to succinate-consuming bacteria in the stool sample from the subject after the targeted intervention lower than the ratio of succinate-producing bacteria to succinate-consuming bacteria in the stool sample from the subject before the targeted intervention is indicative that the targeted intervention has been effective,

and wherein
a ratio of succinate-producing bacteria to succinate-consuming bacteria in the stool sample from the subject after the targeted intervention equal to or higher than the ratio of succinate-producing bacteria to succinate-consuming bacteria in the stool sample from the subject before the targeted intervention is indicative that the targeted intervention has not been effective.

In a particular embodiment, the subject is obese. In another particular embodiment, the subject has type 2 diabetes mellitus. In yet another particular embodiment, the subject is obese and has type 2 diabetes mellitus.

According to the present invention, the ratio of succinate-producing bacteria to succinate-consuming bacteria in the stool sample from the subject after the targeted intervention is considered to be lower than the ratio of succinate-producing bacteria to succinate-consuming bacteria in the stool sample from the subject before the targeted intervention when the ratio of succinate-producing bacteria to succinate-consuming bacteria in the stool sample from the subject after the targeted intervention is at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 1 10%, at least 120%, at least 130%, at least 140%, at least 150% or more lower than the ratio of succinate-producing bacteria to succinate-consuming bacteria in the stool sample from the subject before the targeted intervention.

Likewise, in the context of the present invention, the ratio of succinate-producing bacteria to succinate-consuming bacteria in the stool sample from the subject after the targeted intervention is considered to be higher than the ratio of succinate-producing bacteria to succinate-consuming bacteria in the stool sample from the subject before the targeted intervention when the ratio of succinate-producing bacteria to succinate-consuming bacteria in the stool sample from the subject after the targeted intervention is at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 110%, at least 120%, at least 130%, at least 140%, at least 150% or more higher than the ratio of succinate-producing bacteria to succinate-consuming bacteria in the stool sample from the subject before the targeted intervention.

In the context of the present invention, the targeted intervention directed at reducing the levels of circulating succinate in a subject has been effective, when the levels of circulating succinate in the subject after the targeted intervention are at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 1 10%, at least 120%, at least 130%, at least 140%, at least 150% or more lower than the levels of circulating succinate in the subject before the targeted intervention.

Similarly, the targeted intervention directed at reducing the levels of circulating succinate in a subject has not been effective, when the levels of circulating succinate in the subject after the targeted intervention are equal to or at least 1.5%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%: at least 85%, at least 90%, at least 95%, at least 100%, at least 1 10%, at least 120%, at least 130%, at least 140%, at least 150% or more higher than the levels of circulating succinate in the subject before the targeted intervention.

The expression “intervention directed at reducing the levels of circulating succinate in a subject” refers to any acts realized in a subject with the aim to reduce the levels of circulating succinate in said subject. Preferably, said interventions comprise the administration of specific compounds or combinations of compounds, such as specific nutrients or combinations of nutrients, pharmaceutical or biological compounds. In a particular embodiment, the targeted intervention is selected from the group consisting of a dietary intervention or diet product, a pharmacological intervention, and a probiotic intervention.

Targeted Interventions of the Invention

The targeted intervention may consist of a dietary intervention or diet product. Thus, in yet a further aspect, the invention relates to a dietary intervention or diet product for use in the prevention and/or treatment of a disease associated with increased levels of circulating succinate in a patient, wherein the intervention decreases the ratio of succinate-producing bacteria to succinate-consuming bacteria in the intestinal tract of the patient.

In another aspect, the invention relates to a use of a dietary intervention or diet product for the manufacture of a medicament in the prevention and/or treatment of a disease associated with increased levels of circulating succinate in a patient, wherein the intervention decreases the ratio of succinate-producing bacteria to succinate-consuming bacteria in the intestinal tract of the patient.

In yet another aspect, the invention relates to a method for the treatment and/or prevention of a disease associated with increased levels of circulating succinate in a patient, wherein the method comprises subjecting the patient to a dietary intervention or providing the subject with a diet product, wherein the intervention decreases the ratio of succinate-producing bacteria to succinate-consuming bacteria in the intestinal tract of the patient.

In still another aspect, the invention relates to a use of a dietary intervention or diet product for the prevention and/or treatment of a disease associated with increased levels of circulating succinate in a patient, wherein the intervention decreases the ratio of succinate-producing bacteria to succinate-consuming bacteria in the intestinal tract of the patient.

In another aspect, the invention relates to a dietary intervention or diet product for use in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the intervention decreases the ratio of succinate-producing bacteria to succinate-consuming bacteria in the intestinal tract of the patient.

In another aspect, the invention relates to a use of a dietary intervention or diet product for the manufacture of a medicament in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the intervention decreases the ratio of succinate-producing bacteria to succinate-consuming bacteria in the intestinal tract of the patient.

In yet another aspect, the invention relates to a method for the treatment and/or prevention of a disease selected from the group consisting of obesity, cardiovascular disease, hypertension, type 2 diabetes mellitus, chronic heart failure, ischemic heart disease, ischemia/reperfusion injury and diabetic nephropathy, wherein the method comprises subjecting the patient to a dietary intervention or diet product, wherein the intervention decreases the ratio of succinate-producing bacteria to succinate-consuming bacteria in the intestinal tract of the patient.

In still another aspect, the invention relates to a use of a dietary intervention or diet product for the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the method comprises subjecting the patient to a dietary intervention, wherein the intervention decreases the ratio of succinate-producing bacteria to succinate-consuming bacteria in the intestinal tract of the patient.

As used herein, the term “prevention” refers to the prophylaxis of a disease or condition, i.e., the impediment or hindering of a disease or condition to develop or even to occur, at its initial stage or before its onset. The term “treatment”, as used herein, refers to the eradication, removal, reversion, alleviation, modification, or control of the progression of a disease or condition after its onset and before or after the clinical signs had appeared. More precisely, the progression of the disease or condition is understood to be controlled, if beneficial or desired clinical results appear, including, but not limited to, reduction of symptoms, reduction of the length of the disease or condition, stabilization of the pathological state associated to the condition or disease (specifically avoidance of further deterioration), delay in the disease's or condition's progression, and/or improvement of the pathological state associated with the disease or condition and its remission (both partial and total).

In a particular embodiment, the patient is an obese patient. In another particular embodiment, the subject has type 2 diabetes mellitus. In yet another particular embodiment, the subject is obese and has type 2 diabetes mellitus. The term “obese”, as used herein, refers to a subject that suffers obesity, wherein the term “obesity”, as used herein, is defined as indicated by the World's Health Organization (WHO). According to the WHO, obesity and overweight refer to a condition wherein the subject with obesity or overweight has an abnormal or excessive fat accumulation that may impair health. More specifically, WHO defines overweight and obesity using the body mass index (BMI) as follows, wherein the BMI is defined as a person's weight in kilograms divided by the square of his height in meters (kg/m2):
overweight is a condition of a subject with a BMI greater than or equal to 25 kg/m2;
obesity is a condition of a subject with a BMI greater than or equal to 30 kg/m2.

The expression “disease associated with increased levels of circulating succinate in a patient”, as used herein, refers to a disease known to be concurrent with elevated levels of circulating succinate, which have been associated with the progression of the disease. Non limiting examples of such diseases are hypertension (Sadagopan et al., 2007, Am. J. Hypertens. 20:1209-1215), ischemic heart disease (Aguiar et al., 2014, Cell Commun. Signal. 12:78), type 2 diabetes mellitus (T2DM) (Guo et al., 2017, Nat. Commun. 8:15621; Sadagopan et al., 2007, Am. J. Hypertens. 20:1209-1215; Toma et al., 2008, J. Clin. Invest. 118:2526-2534; van Diepen et al., 2017, Diabetologia 60:1304-1313), hypertrophic cardiomyopathy (Aguiar et al., 2014, Cell Commun. Signal. 12:78), obesity-related metabolic disturbances (McCreath et al., Diabetes. 2015 April; 64(4):1154-67), renin-induced hypertension (Toma et al., 2008, J. Clin. Invest. 118:2526-2534), and diabetic retinopathy (Ariza et al., 2012, Front. Endocrinol. (Lausanne) 3: 22). In addition, elevated succinate levels have been described in autoimmune diseases. Succinate has been abundantly detected in synovial fluid (SF) from rheumatoid arthritis (RA) patients (Borestein et al., 1982, Arthritis Rheum. 25:947-953; Kim et al., 2014, PLoS One. 9:e97501), and a metabolic profiling study has identified succinate as the most differentially expressed metabolite in RA compared with other arthropathies (Kim et al., 2014, PLoS One. 9:e97501).

In a particular embodiment, the disease associated with increased levels of circulating succinate in a patient is selected from the group consisting of obesity, cardiovascular disease, hypertension, type 2 diabetes mellitus, chronic heart failure, ischemic heart disease, ischemia/reperfusion injury and diabetic nephropathy. In a preferred embodiment, the disease is obesity.

The expression “increased levels of circulating succinate”, as used herein, refers to a level of circulating succinate increased with respect to a reference value. In a particular embodiment the reference value is obtained from a healthy subject. In another particular embodiment the reference value is obtained from subjects who do not have a clinical history of a disease associated with increased levels of circulating succinate, preferably of the diseases listed above.

In a particular embodiment, it is considered that the levels of circulating succinate are increased with respect to the levels of circulating succinate in a reference sample when it increases in at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 100%, at least 120%, at least 150%, at least 200% or more in respect to a reference sample.

The expression “decreases the ratio of succinate-producing bacteria to succinate-consuming bacteria”, as used herein, refers to interventions wherein the ratio of succinate-producing bacteria to succinate-consuming bacteria decreases either by decreasing the total amount of succinate-producing bacteria, by increasing the total amount of succinate-consuming bacteria, or by a combination of both decreasing the total amount of succinate-producing bacteria and increasing the total amount of succinate-consuming bacteria.

The term “intestinal tract of the patient”, or “gastrointestinal tract”, as used herein, generally refers to the digestive structures stretching from the mouth to the anus, but does not include the accessory glandular organs such as the liver; the biliary tract; or the pancreas. The gastrointestinal tract is an organ system within humans and other animals which takes in food, digests it to extract and absorb energy and nutrients, and expels the remaining waste as feces. The mouth, oesophagus, stomach, small intestine, and large intestine are part of the gastrointestinal tract. The gastrointestinal tract contains thousands of different bacteria in its gut flora. In a particular embodiment, the term “gastrointestinal tract” in the context of the present invention refers specifically to the small intestine and/or the large intestine.

In a particular embodiment, the ratio of succinate-producing bacteria to succinate-consuming bacteria to be reduced is the (Prevotellaceae+Veillonellaceae)/(Odoribacteriaceae+Clostridiaceae) ratio.

The expression “dietary intervention”, as used herein, refers to an act, or group of acts, realized in a subject that comprise following a specific diet of interest. The term “diet product”, as used herein, refers to a complete set of meals to be provided to the subject and which makes up at least 60%, at least 70%, at least 80%, at least 90%, at least 95%, at least 99%, or 100% of the total food intake of said subject. In a particular embodiment, the diet product comprises at least breakfast, lunch and dinner. In a particular embodiment, the diet product comprises one daily meal, two daily meals, three daily meals, four daily meals, or five or more daily meals. The term “diet”, as used herein, refers to an indication on the food that a subject has to consume in order to achieve the absorption of a pre-determined amount of specific nutrients. In some cases, the diet may also comprise an indication on the type and amount of liquid to be consumed by the subject and on a type and duration of physical exercise that the subject has to realize in order to achieve said absorption of a pre-determined amount of specific nutrients. Non-limiting examples of diet are a hypocaloric diet or a Mediterranean diet.

In a particular embodiment, the intervention comprises a hypocaloric diet, preferably a hypocaloric diet characterized in that:
fat is 35-40% of total daily calorie intake; and
carbohydrates are 40-45% of total daily calorie intake;

wherein the dietary intervention or diet product is administered for at least 12 weeks, and wherein the dietary intervention or diet product is optionally administered in combination with a physical exercise program.

The expression “total daily calorie intake”, as used herein, refers to the result of adding the calories of each food item ingested by the subject during one given day. To calculate the daily fat percentage, calories from fat are divided by total calories and then multiplied by 100. To calculate the daily carbohydrate percentage, calories from carbohydrates are divided by total calories and then multiplied by 100.

In a preferred embodiment, the diet is a Mediterranean diet. In the context of the present invention, a “Mediterranean diet” is characterized in that it includes proportionally high consumption of olive oil, legumes, unrefined cereals, fruits, and vegetables, moderate to high consumption of fish, moderate consumption of dairy products (mostly as cheese and yogurt), moderate wine consumption, and low consumption of non-fish meat products. In a preferred embodiment, the diet includes extra virgin olive oil and nuts. In a preferred embodiment, 8-10% of total fat are saturated fatty acids. In a preferred embodiment, the carbohydrates are of low glycemic index. Low glycemic index carbohydrates are characterized by a GI range of 55 or less. Examples of low GI carbohydrates are fructose; beans (black, pinto, kidney, lentil, peanut, chickpea); small seeds (sunflower, flax, pumpkin, poppy, sesame, hemp); walnuts, cashews, most whole intact grains (durum/spelt/kamut wheat, millet, oat, rye, rice, barley); most vegetables, most sweet fruits (peaches, strawberries, mangos); tagatose; mushrooms; and chilis. In a preferred embodiment, protein is 20% of total daily calorie intake.

In a preferred embodiment the dietary intervention or diet product is administered for at least 12 weeks, for at least 16 weeks, for at least 20 weeks, for at least 24 weeks, for at least one year, for at least two years, for at least three years, for at least four years, for at least five years, or indefinitely. In a preferred embodiment wherein the dietary intervention or diet product is optionally administered in combination with a physical exercise program, the duration of physical exercise is of at least 45 minutes a day during the duration of the administration of the diet product.

In the context of the present invention, a “hypocaloric diet” is a diet wherein the subject eats fewer calories than he or she spends throughout the day. Accordingly, in order to design a hypocaloric diet, the need for daily calories of the subject needs to be calculated: i.e. the basal metabolic expenditure has to be determined (the expense that the body makes for normal functioning) and adding the calories what the subject spends through daily physical activity (i.e. walking, climbing stairs, etc.) and sports activity (i.e. training).

Basal metabolic expenditure may be calculated using different methods and it depends on different factors, such as the height and weight of each person. It is also influenced by factors such as age, muscle mass, body temperature, etc. Basic metabolic expenditure may be calculated for example using the Harris-Benedict equation:
Basal metabolism in men (metric): 66,473+(13,751×weight in kg)+(5,0033×height in cm)−(6,7550×age in years)
Basal metabolism in women (metric): 655.1+(9.463×weight in kg)+(1.8×height in cm)−(4.6756×age in years)

Thus, in a preferred embodiment the daily calorie intake is reduced by 200 kcal with respect to total daily calorie needs; in a preferred embodiment the daily calorie intake is reduced by 300 kcal with respect to total daily calorie needs; in a preferred embodiment the daily calorie intake is reduced by 400 kcal with respect to total daily calorie needs; in a preferred embodiment the daily calorie intake is reduced by 500 kcal with respect to total daily calorie needs; in a preferred embodiment the daily calorie intake is reduced by 600 kcal with respect to total daily calorie needs.

The targeted intervention may also refer to pharmaceutical intervention or to a probiotic intervention. Thus, in an aspect, the invention relates to a product for use in the prevention and/or treatment of a disease associated with increased levels of circulating succinate in a patient, wherein the product decreases the ratio of succinate-producing bacteria to succinate-consuming bacteria in the intestinal tract of the patient, wherein the product is selected from the group consisting of a pharmacological product, and a probiotic product. In a particular aspect, the invention also relates to a product for use in the prevention and/or treatment of a disease associated with increased levels of circulating succinate in a patient, wherein the product decreases the levels of circulating succinate of the patient, wherein the product is selected from the group consisting of a pharmacological product, and a probiotic product.

In another aspect, the invention relates to a use of a pharmacological product or a probiotic product for the manufacture of a medicament in the prevention and/or treatment of a disease associated with increased levels of circulating succinate in a patient, wherein the intervention decreases the ratio of succinate-producing bacteria to succinate-consuming bacteria in the intestinal tract of the patient. In a particular aspect, the invention also relates to a use of a pharmacological product or a probiotic product for the manufacture of a medicament in the prevention and/or treatment of a disease associated with increased levels of circulating succinate in a patient, wherein the product decreases the levels of circulating succinate of the patient.

In yet another aspect, the invention relates to a method for the treatment and/or prevention of a disease associated with increased levels of circulating succinate in a patient, wherein the method comprises administering a pharmacological product or a probiotic product to the patient, wherein the intervention decreases the ratio of succinate-producing bacteria to succinate-consuming bacteria in the intestinal tract of the patient. In a particular aspect, the invention also relates to a method for the treatment and/or prevention of a disease associated with increased levels of circulating succinate in a patient, wherein the method comprises administering a pharmacological product or a probiotic product to the patient, wherein the product decreases the levels of circulating succinate of the patient.

In another aspect, the invention relates to a product for use in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the product decreases the ratio of succinate-producing bacteria to succinate-consuming bacteria in the intestinal tract of the patient, wherein the product is selected from the group consisting of a pharmacological product, and a probiotic product. In a particular aspect, the invention also relates to a product for use in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the product decreases the levels of circulating succinate of the patient, wherein the product is selected from the group consisting of a pharmacological product, and a probiotic product.

In another aspect, the invention relates to a use of a pharmacological product or a probiotic product for the manufacture of a medicament in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the intervention decreases the ratio of succinate-producing bacteria to succinate-consuming bacteria in the intestinal tract of the patient. In a particular aspect, the invention also relates to a use of a pharmacological product or a probiotic product for the manufacture of a medicament in the prevention and/or treatment of a disease selected from the group consisting of obesity, cardiovascular disease, hypertension, type 2 diabetes mellitus, chronic heart failure, ischemic heart disease, ischemia/reperfusion injury and diabetic nephropathy, wherein the product decreases the levels of circulating succinate of the patient.

In yet another aspect, the invention relates to a method for the treatment and/or prevention of a disease selected from the group consisting of obesity, cardiovascular disease, hypertension, type 2 diabetes mellitus, chronic heart failure, ischemic heart disease, ischemia/reperfusion injury and diabetic nephropathy, wherein the method comprises administering a pharmacological product or a probiotic product to the patient, wherein the intervention decreases the ratio of succinate-producing bacteria to succinate-consuming bacteria in the intestinal tract of the patient. In a particular aspect, the invention also relates to a method for the treatment and/or prevention of a disease selected from the group consisting of obesity, cardiovascular disease, hypertension, type 2 diabetes mellitus, chronic heart failure, ischemic heart disease, ischemia/reperfusion injury and diabetic nephropathy, wherein the method comprises administering a pharmacological product or a probiotic product to the patient, wherein the product decreases the levels of circulating succinate of the patient.

The terms and expressions “prevention”, “treatment”, “decreases the ratio of succinate-producing bacteria to succinate-consuming bacteria”, “decreases the levels of circulating succinate”, and “intestinal tract” are used as defined above.

The term “pharmacological intervention”, as used herein, refers to an act, or group of acts, realized in a subject that comprise administering a pharmacological product of interest to a subject. The expression “pharmacological product”, or “pharmacological composition”, as used herein, refers a product or composition with a chemical formulation that has been adapted for administering a predetermined dose of one or several therapeutic agents for the treatment of a specific disease or condition. Said agents are typically in combination with a pharmaceutically acceptable carrier in said pharmacological product or composition. The term “carrier”, as used herein, refers to a diluent or excipient with which the active ingredient, or active agent, is administered. Such pharmaceutical carriers can be sterile liquids, such as water and oils, including those of petroleum, animal, vegetable or synthetic origin, such as peanut oil, soybean oil, mineral oil, sesame oil and similar. These are preferably employed as water carriers or saline aqueous solutions and aqueous dextrose and glycerol solutions, particularly for injectable solutions. Suitable pharmaceutical carriers are described in “Remington's Pharmaceutical Sciences” by E W Martin, 1995. Preferably, the invention carriers are approved by the regulatory agency of a state of federal government or are listed in the U.S. Pharmacopeia or other generally recognized pharmacopeia for use in animals, and more particularly in humans. The vehicles and auxiliary substances necessary to manufacture the desired pharmaceutical form of administration of the pharmaceutical composition of the invention will depend, among other factors, on the selected pharmaceutical form of administration. Said pharmaceutical forms of administration of the pharmaceutical composition will be manufactured according to conventional methods known to the skilled artisan. A review of different methods of administration of active principles, excipients to be used and procedures to produce them can be found in “Tratado de Farmacia Galénica”, C. Fauli i Trillo, Luzán 5, S. A. de Ediciones, 1993. Examples of pharmaceutical compositions include any solid composition (tablets, pills, capsules, granules, etc.) or liquid (solutions, suspensions or emulsions) for oral, topical or parental administration. Furthermore, the pharmaceutical composition may contain stabilizers, suspensions, preservatives, surfactants and the like as necessary.

The term “probiotic intervention”, as used herein, refers to an act, or group of acts, realized in a subject that comprise administering a probiotic product of interest to a subject. The expression “probiotic product”, or “probiotic composition”, as used herein, refers a product or composition with a probiotic agent, wherein probiotic agent is understood as live microorganisms which provide health benefit on the host when administered in adequate amounts. Probiotics exhibit their beneficial effects when they are alive. Preferably, said health benefits are specific for, and even more preferably, they are the basis of the treatment or prevention of a specific disease or condition. Typically, probiotics are bacterial populations. There are four basic ways for consuming probiotics: as a concentrated culture added to a drink (e.g., fruit juice, etc.), inoculated in prebiotic fibers, as a dietary supplement in lyophilized cell dosage forms (e.g., powder, capsules, tablets, etc.) and inoculated in milk-based foods.

In a particular embodiment, the patient is an obese patient. The term “obese” is used as defined above. In a particular embodiment, the disease associated with increased levels of circulating succinate in a patient is selected from the group consisting of obesity, cardiovascular disease, hypertension, type 2 diabetes mellitus, chronic heart failure, ischemic heart disease, ischemia/reperfusion injury and diabetic nephropathy.

In a particular embodiment, the ratio of succinate-producing bacteria to succinate-consuming bacteria to be decreased is the (Prevotellaceae+Veillonellaceae)/(Odoribacteriaceae+Clostridiaceae) ratio.

In a particular embodiment, the pharmacological product specifically targets succinate producing bacteria, and preferably wherein the pharmacological product is selected from the group consisting of an antibiotic, an antibacterial antibody, and a bacteriophage. The expression “specifically targets succinate producing bacteria” refers to a pharmacological product that selectively reduces the population of succinate producing bacteria in relation to total bacteria. Screening methods to determine whether a pharmacological product selectively reduces the population of succinate producing bacteria may be readily designed by the person skilled in the art. In a particular example, a screening method to determine whether a specific pharmacological product selectively reduces the population of succinate producing bacteria may comprise culturing Prevotella in a medium containing the specific pharmacological product and comparing the growth of Prevotella with the growth of other types of bacteria.

The term “antibiotic”, as used herein, refers to are a type of antimicrobial product or composition used in the treatment and prevention of bacterial infections. They may either kill or inhibit the growth of bacteria. They can be administered in the form of a pharmacological product or composition. In a particular embodiment, the antibiotic is an antibiotic specific against gram-negative bacteria. In a preferred embodiment, the antibiotic specific against gram-negative bacteria is a β-lactam antibiotic. In a more preferred embodiment, the antibiotic specific against gram-negative bacteria is a monobactam antibiotic. In a still more preferred embodiment, the antibiotic specific against gram-negative bacteria is aztreonam.

The term “antibacterial antibody” as used herein refers to (a) an antibody-antibiotic conjugate (AAC) that combines key attributes of an antibody and antibiotic, or (b) an antibacterial monoclonal antibody (DiGiandomenico and Sellman, Current Opinion in Microbiology 2015, 27: 78-85). An AAC has three components: an antibiotic payload to kill bacteria, an antibody to target delivery of the payload to bacteria, and a linker attaching the payload to the antibody. AAC are potentially efficient in treating specific bacterial infections. A Non-limiting example of bacteria that has been shown to be targeted by an AAC is Staphylococcus aureus. Antibacterial monoclonal antibody technology on the other hand refers to the use of bacteria specific monoclonal antibodies (mAbs) to reduce the bacterial load of said specific bacteria. Passive immunization of individuals with mAbs selected for superior functional activity may both neutralize bacterial virulence and take advantage of the host immune response against the specific bacteria. In this sense, bacterial capsular polysaccharides have been successfully targeted as vaccine antigens (i.e. against Streptococcus pneumoniae and Haemophilus influenzae) but specific antitoxin antibodies are also being developed. Surface antigens are considered a promising target for antibacterial antibody discovery. The key activities of bacterial surface-specific mAbs are engagement of the host immune system through complement fixation and opsonophagocytic killing (OPK). The general methodology for making monoclonal antibodies by hybridomas is well known. Immortal, antibody-producing cell lines can also be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies And T-cell Hybridomas” (1981); Kennett et al., “Monoclonal Antibodies” (1980). Monoclonal antibodies against Prevotella are commercially available (i.e. anti-Prevotella intermedia monoclonal antibody DMAB9450 against Prevotella intermedia strain OMZ 248 from human chronic periodontitis, by Creative Diagnostics).

The term “bacteriophage”, as used herein, refers to a virus that infects and replicates within a bacterium. Phages replicate within the bacterium following the injection of their genome into its cytoplasm. They have been used for over 90 years as an alternative to antibiotics. In a particular embodiment, the bacteriophage selectively infects Prevotella. In a preferred embodiment, the bacteriophage selectively infects Prevotella ruminicola. In a more preferred embodiment, the bacteriophage is selected from the group consisting of ϕBRB01, ϕBRB02 (Klieve et al. 1989 Apl. Environ. Microbiol. 55: 1630-4) and ϕ4AR29 (Gregg K et. al. 1994 Microbiology 140: 2109-14). In another preferred embodiment, the bacteriophage selectively infects Bacteroides fragilis, which is also a known succinate producer. In a more preferred embodiment, the bacteriophage is selected from the group consisting of ϕB124-14 and ϕB40-8 (Ogilvie et al. 2013 Nature Communications 4, 2420).

In a particular embodiment, the probiotic product comprises succinate consuming bacteria. In a preferred embodiment, the succinate consuming bacteria is selected from the group consisting of Odoribacter spp, Clostridium spp, Phascolarctobacterium spp, Ruminococcus spp, and combinations thereof.

In a particular embodiment, the probiotic product is a combination of Odoribacter spp. and Clostridium spp.; a combination of Odoribacter spp. and Ruminococcus spp.; a combination of Odoribacter spp. and Phascolarctobacterium spp.; a combination of Clostridium spp., and Ruminococcus spp.; a combination of Clostridium spp, and Phascolarctobacterium spp.; a combination of Ruminococcus spp., and Phascolarctobacterium spp.; a combination of Odoribacter spp., Clostridium spp. and Ruminococcus spp.; a combination of Odoribacter spp., Clostridium spp., and Phascolarctobacterium spp.; a combination of Odoribacter spp., Ruminococcus spp., and Phascolarctobacterium spp.; a combination of Clostridium spp., Ruminococcus spp. and Phascolarctobacterium spp.; or a combination of Odoribacter spp., Clostridium spp., Ruminococcus spp., and Phascolarctobacterium spp.

In a preferred embodiment, the Odoribacter spp. is selected from the group consisting of Odoribacter laneus; Odoribacter splanchnicus and combinations thereof. In a more preferred embodiment the Odoribacter spp. is Odoribacter laneus. In a still more preferred embodiment the Odoribacter spp. is Odoribacter laneus type strain DSM22474. In a preferred embodiment, the Clostridium spp. is selected from the group consisting of Clostridium scindens; Clostridium symbiosum; Clostridium perfringens; Clostridium citroniae; Clostridium hathewayi; Clostridium ramosum and combinations thereof. In a preferred embodiment, the Phascolarctobacterium spp. is selected from the group consisting of Phascolarctobacterium succinatutens and Phascolarctobacterium faecium. In a preferred embodiment, the Ruminococcus spp. is Ruminococcus bromii.

In another aspect, the invention relates to a product for use in a method for improving an altered metabolic profile in a patient, wherein the product decreases the ratio of succinate-producing bacteria to succinate-consuming bacteria in the intestinal tract of a patient, and wherein the product is selected from the group consisting of a diet product, a pharmacological product, and a probiotic product. All the terms and embodiments described elsewhere herein are equally applicable to this aspect of the invention.

The term “altered metabolic profile”, as used herein refers to a set of threshold values for a number of parameters which are associated with the risk of developing metabolic pathologies such as diabetes. In a particular embodiment, the altered metabolic profile is associated with an increased risk of suffering from a metabolic dysfunction selected from the group consisting of obesity, cardio vascular disease, hypertension, type 2 diabetes mellitus, chronic heart failure, ischemic cardiomyopathy, ischemia/reperfusion injury and diabetic nephropathy. The values characteristic of an altered metabolic profile in the context of the present invention are as follows:
Insulin >25 μLU/mL
Glucose >100 (mg/dl)
HOMA-IR >3.21
Triglycerides >1.7 (mM)

The threshold values for glucose and triglycerides are values defined by the American Diabetes Association, American Heart Association or by the International Diabetes Federation in order to define the metabolic syndrome. However, in the context of the invention these thresholds do not necessarily relate to Metabolic Syndrome. The threshold value for HOMA-IR (Homeostasis model assessment of insulin resistance index) has been described elsewhere (Ceperuelo-Mallafré et al., J Clin Endocrinol Metab. 2014 May; 99(5): E908-19; Cardona F. et al., Clin Chem. 2006 October; 52(10): 1920-5).

The term “improving an altered metabolic profile in a patient”, as used herein refers to acts directed at decreasing the values corresponding to the parameters associated with the risk of developing metabolic pathologies such as diabetes. In a particular embodiment, the values corresponding to the parameters associated with the risk of developing metabolic pathologies are decreased below the threshold values that define the altered metabolic profile. In a preferred embodiment, all values corresponding to the parameters associated with the risk of developing metabolic pathologies are decreased below the threshold values that define the altered metabolic profile.

In a final aspect, the invention relates to a probiotic product comprising an effective amount of succinate consuming bacteria, wherein the succinate consuming bacteria is selected from the group consisting of Odoribacter spp, Phascolarctobacterium spp, Ruminococcus spp, and combinations thereof. The terms “probiotic product” and “succinate consuming bacteria” are used as defined above.

In a particular embodiment, the probiotic product is a combination of Odoribacter spp. and Clostridium spp.; a combination of Odoribacter spp. and Ruminococcus spp.; a combination of Odoribacter spp. and Phascolarctobacterium spp.; a combination of Clostridium spp., and Ruminococcus spp.; a combination of Clostridium spp, and Phascolarctobacterium spp.; a combination of Ruminococcus spp., and Phascolarctobacterium spp.; a combination of Odoribacter spp., Clostridium spp. and Ruminococcus spp.; a combination of Odoribacter spp., Clostridium spp., and Phascolarctobacterium spp.; a combination of Odoribacter spp., Ruminococcus spp., and Phascolarctobacterium spp.; a combination of Clostridium spp., Ruminococcus spp. and Phascolarctobacterium spp.; or a combination of Odoribacter spp., Clostridium spp., Ruminococcus spp., and Phascolarctobacterium spp.

In a preferred embodiment, the Odoribacter spp. is selected from the group consisting of Odoribacter laneus; Odoribacter splanchnicus and combinations thereof. In a more preferred embodiment the Odoribacter spp. is Odoribacter laneus. In a still more preferred embodiment the Odoribacter spp. is Odoribacter laneus type strain DSM22474. In a preferred embodiment, the Clostridium spp. is selected from the group consisting of Clostridium scindens; Clostridium symbiosum; Clostridium perfringens; Clostridium citroniae; Clostridium hathewayi; Clostridium ramosum and combinations thereof. In a preferred embodiment, the Phascolarctobacterium spp. is selected from the group consisting of Phascolarctobacterium succinatutens and Phascolarctobacterium faecium. In a preferred embodiment the Ruminococcus spp. is Ruminococcus bromii.

The invention further discloses the following aspects:
1. A kit comprising reagents suitable for determining the ratio of succinate-producing bacteria to succinate-consuming bacteria in a stool sample from a subject,
   wherein the kit comprises primer sets designed to specifically hybridize the hypervariable regions of the 16S rRNA gene in at least one succinate-producing bacterium and in at least one succinate-consuming bacterium, or
   wherein the kit comprises probes that specifically hybridize to the hypervariable regions of the 16S rRNA gene in at least one succinate-producing bacterium and in at least one succinate-consuming bacterium,

and wherein the primer sets or the probes comprise at least 10% of the total amount of reagents forming the kit.
2. Use of the kit according to aspect 1 to detect the ratio of succinate-producing bacteria to succinate-consuming bacteria in a stool sample from a subject.
3. The kit according to aspect 1, or the use according to aspect 2, wherein the ratio of succinate-producing bacteria to succinate-consuming bacteria is the (Prevotellaceae+Veillonellaceae)/(Odoribacteriaceae+Clostridiaceae) ratio.
4. A method for determining whether a targeted intervention directed at reducing the levels of circulating succinate in a subject has been effective, the method comprising:
   (a) determining the ratio of succinate-producing bacteria to succinate-consuming bacteria in a stool sample from the subject before the targeted intervention, and
   (b) determining the ratio of succinate-producing bacteria to succinate-consuming bacteria in a stool sample from the subject after the targeted intervention,

wherein
a ratio of succinate-producing bacteria to succinate-consuming bacteria in the stool sample from the subject after the targeted intervention lower than the ratio of succinate-producing bacteria to succinate-consuming bacteria in the stool sample from the subject before the targeted intervention is indicative that the targeted intervention has been effective,

and wherein
a ratio of succinate-producing bacteria to succinate-consuming bacteria in the stool sample from the subject after the targeted intervention equal to or higher than the ratio of succinate-producing bacteria to succinate-consuming bacteria in the stool sample from the subject before the targeted intervention is indicative that the targeted intervention has not been effective.
5. The method of aspect 4, wherein the targeted intervention is selected from the group consisting of a dietary intervention or diet product, a pharmacological intervention, and a probiotic intervention.
6. A dietary intervention or diet product for use in the prevention and/or treatment of a disease associated with increased levels of circulating succinate in a patient, wherein the intervention decreases the ratio of succinate-producing bacteria to succinate-consuming bacteria in the intestinal tract of the patient.
7. The dietary intervention or diet product for use according to aspect 6, wherein the intervention comprises a hypocaloric diet characterized in that:
   fat is 35-40% of total daily calorie intake; and
   carbohydrates are 40-45% of total daily calorie intake;

wherein the dietary intervention or diet product is administered for at least 12 weeks, and

wherein the dietary intervention or diet product is optionally administered in combination with a physical exercise program.
8. A product for use in the prevention and/or treatment of a disease associated with increased levels of circulating succinate, wherein the product decreases the ratio of succinate-producing bacteria to succinate-consuming bacteria in the intestinal tract of the patient, wherein the product is selected from the group consisting of a pharmacological product, and a probiotic product.
9. The dietary intervention or diet product for use according to any one of aspects 6 or 7, or the product for use according to aspect 8, wherein the patient is obese.
10. The dietary intervention or diet product for use according to any one of aspects 6, 7 or 9, or the product for use according to any one of aspects 8 or 9, wherein the disease associated with increased levels of circulating succinate in a patient is selected from the group consisting of obesity, cardiovascular disease, hypertension, type 2 diabetes mellitus, chronic heart failure, ischemic heart disease, ischemia/reperfusion injury and diabetic nephropathy.
11. The dietary intervention or diet product for use according to any one of aspects 6-7, or 9-10, or the product for use according to any one of aspects 8 to 10, wherein the ratio of succinate-producing bacteria to succinate-consuming bacteria is the (Prevotellaceae+Veillonellaceae)/(Odoribacteriaceae+Clostridiaceae) ratio.
12. The product for use according to any one of aspects 8 to 11, wherein the pharmacological product specifically targets succinate producing bacteria, and wherein the pharmacological product is selected from the group consisting of an antibiotic, an antibacterial antibody, and a bacteriophage.
13. The product for use according to any one of aspects 8 to 12, wherein the probiotic product comprises succinate consuming bacteria.
14. The product for use according to aspect 13, wherein the succinate consuming bacteria is selected from the group consisting of Odoribacter spp, Clostridium spp, Phascolarctobacterium succinatutens, and combinations thereof.
15. A probiotic product comprising an effective amount of succinate consuming bacteria, wherein the succinate consuming bacteria is selected from the group consisting of Odoribacter spp, Clostridium spp, Phascolarctobacterium succinatutens, and combinations thereof.

The following invention is hereby described by way of the following examples, which are to be construed as merely illustrative and not limitative of the scope of the invention.

### EXAMPLES

Materials and Methods

Study Design and Patients

The present study comprised five different clinical sub-studies to serve the following different aims: 1) analyze circulating succinate levels in lean, obese and diabetic subjects using a cross-sectional study, cohort I; 2) examine the relationship between gut microbiota and succinate (discovery cohort II and confirmatory cohort III); 3) establish a link between circulating succinate and gut microbiota (dietetic intervention study cohort IV and follow-up study cohort V).

All studies were conducted in accordance to the principles of the Declaration of Helsinki. All volunteers received information concerning their participation in the study and gave written informed consent. The studies were approved by the respective local Ethics Committee review boards of the participating Hospitals.

Inclusion criteria for all subjects: (1) Caucasian men and women; (2) BMI range from lean to obese (adequately represented in each group); (3) absence of underlying pathology on physical examination and tests other than those associated with an excess of weight or diabetes; (4) signed informed consent for participation in the study.

Exclusion criteria for all subjects: (1) serious systemic disease unrelated to obesity such as cancer, severe kidney or liver disease; (2) systemic diseases with intrinsic inflammatory activity; (3) history of liver disease (chronic active hepatitis or cirrhosis) and/or abnormal liver function (ALT and/or AST 3 times above the upper normal value); altered renal function (creatinine greater than 1.4 mg/dl in women and 1.5 mg/dl in men); (4) pregnancy and lactation; (5) vegetarians or subjects subjected to irregular diet; (6) patients with severe disorders of eating behavior; (7) clinical symptoms and signs of infection in the previous month; (8) anti-inflammatory chronic treatment with steroidal and/or non-steroidal anti-inflammatory drugs; (9) prior antibiotic treatment in the last 3 months; (10) major psychiatric antecedents; (11) uncontrolled alcoholism or drug abuse.

Cross-Sectional Study Cohort I

Design: Observational Single-Point Study.

Participants: Ninety-one subjects (49 women and 42 men) were included in the cross-sectional study (30 lean, 41 obese and 20 patients with T2DM). Obesity was classified according to World Health Organization (WHO) criteria. Patients with T2DM were diagnosed according to American Diabetes Association criteria with a stable metabolic control in the previous 6 months, as defined by stable glycosylated hemoglobin values. No patient was insulin treated; 60% were taking metformin, 20% were treated with sulfonylurea and less than 15% were treated with dipeptidyl peptidase-4 inhibitors. Subjects were recruited at the Endocrinology Service at the University Hospital Joan XXIII (Tarragona, Spain).

Intervention: All patients had fasted overnight before collection of subcutaneous adipose tissue (SAT) and blood. SAT was obtained during scheduled non-acute surgical procedures including laparoscopic surgery for hiatus hernia repair or cholecystectomies. SAT samples were washed in phosphate buffered saline (PBS) and immediately frozen in liquid N2 with storage at −80° C., or used immediately for fractionation. For SAT fractionation, fresh SAT was diced into small pieces (10-30 mg), washed in PBS and incubated in Medium 199 (Gibco, Gran Island, N.Y.) plus 4% bovine serum albumin and 2 mg/ml collagenase type I (Sigma-Aldrich, St. Louis, Mo.) for 1 h in a shaking water bath at 37° C. Anthropometrical and clinical variables are summarized in Table 1.

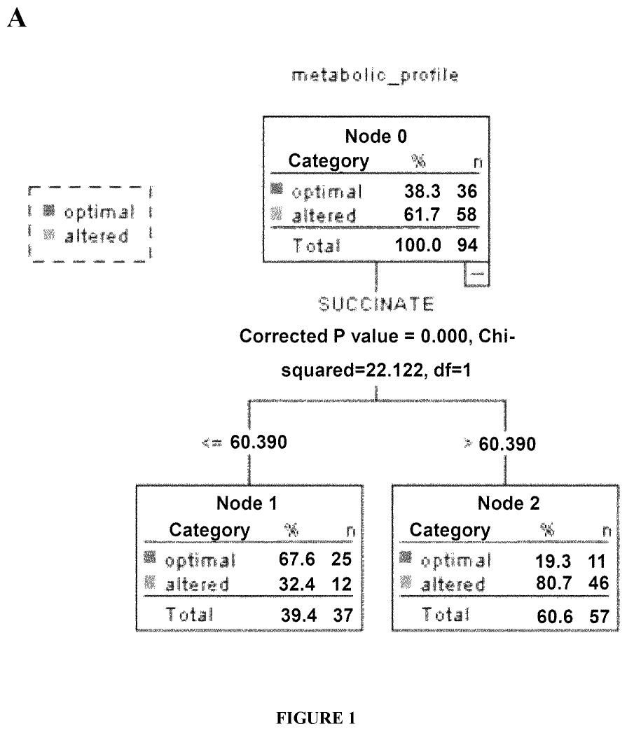

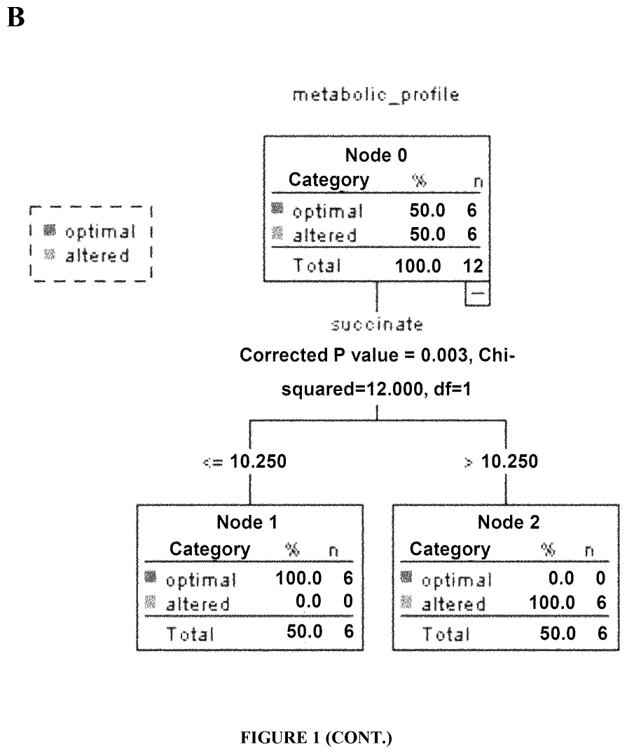

TABLE 1
Anthropometric and analytical characteristics in the Cohort I. Related to FIG. 1.
Cross-sectional study (Cohort I)

| | Lean | Obese | T2DM |
|---|---|---|---|
| n | 30 | 41 | 20 |
| Age (years) | 49 (40.75-63.25) | 56 (41.50-70) | 65 (56.75-70.75) |
| Sex (females/males) | 14/16 | 23/18 | 12/8 |
| BMI (kg/m²) | 23.75 (22-24.88) | 30.52 (27.75-33.66)** | 29.06 (27-31.03) |
| Insulin (μU/ml) | 5.65 (2.55-8.42) | 9.87 (4.45-16.07)* | 13.31 (9.42-21.64) |
| Glucose (mg/dl) | 89 (83-106) | 100 (88.75-108.5)* | 139.5 (109.75-162.75)^{##} |
| HOMA IR | 1.50 (0.98-2.40) | 2.55 (1.19-3.45) | 4.96 (3.11-11.31)^{##} |
| SBP | 119.40 ± 6.07 | 138.13 ± 18.95* | 142.5 ± 20 |
| DBP | 67 (60-75) | 80 (70-80) | 80 (62.5-83) |
| Cholesterol (mM) | 4.86 ± 1.23 | 4.94 ± 1.05 | 4.58 ± 1.08 |
| HDL cholesterol (mM) | 1.35 (1.04-1.61) | 1.23 (1.02-1.52) | 1.11 (0.98-1.47) |
| Tryglycerides (mM) | 0.97 (0.86-1.09) | 1.23 (0.74-1.66) | 1.83 (1.38-2.26)^{#} |
| sIL6 (pg/ml) | 1.62 (1.33-2.77) | 1.63 (1.02-2.57) | 1.70 (1.03-2.44) |
| sTNFR1 (ng/ml) | 1.92 ± 0.53 | 2.02 ± 0.35 | 2.23 ± 0.39 |
| sTNFR2 (ng/ml) | 2.92 ± 0.89 | 3.60 ± 1.17 | 5.43 ± 1.07^{##} |

Data are presented as mean ± SD or median (25th-75th), as appropriate. Differences were analyzed by one-way ANOVA with Bonferroni adjustment (normal distribution) or Kruskal-Wallis test with post hoc Dunn's multiple comparison test (data not-normally distributed). BMI: Body mass index; HOMA-IR: Homeostasis model assessment of insulin resistance index; SBP: Systolic blood pressure; DBP: Diastolic blood pressure. *p < 0.05, **p < 0.01 vs lean; ^{#}p < 0.05, ^{##}p < 0.01 vs obese.

Discovery Cohort H

Design: Observational Single-Point Study.

Participants: Twenty female subjects were included in the cross-sectional study (10 lean and 10 obese). Obesity was classified according to WHO criteria. Subjects were recruited in the outpatient surgery at the Endocrinology Service at the University Hospital Virgen de la Victoria de Milaga (Malaga, Spain). The study participants received no antibiotic treatment, probiotics, prebiotics or any other medical treatment influencing intestinal microbiota during the 3 months before the start of the study.

Intervention: All patients had fasted overnight before collection of blood and stool. Anthropometrical and clinical variables are summarized in Table 2.

Confirmatory Cohort III

Design: Observational Single-Point Study.

Participants: Seventeen subjects (10 women and 7 men) were included in the study (9 lean and 8 obese). Obesity was classified according to WHO criteria. Subjects were recruited at the Endocrinology Service at the University Hospital Dr. Josep Trueta (Girona, Spain). The study participants received no antibiotic treatment, probiotics, prebiotics or any other medical treatment influencing intestinal microbiota during the 3 months before the start of the study.

Intervention: All patients had fasted overnight before collection of blood and stool. Anthropometrical and clinical variables are summarized in Table 2.

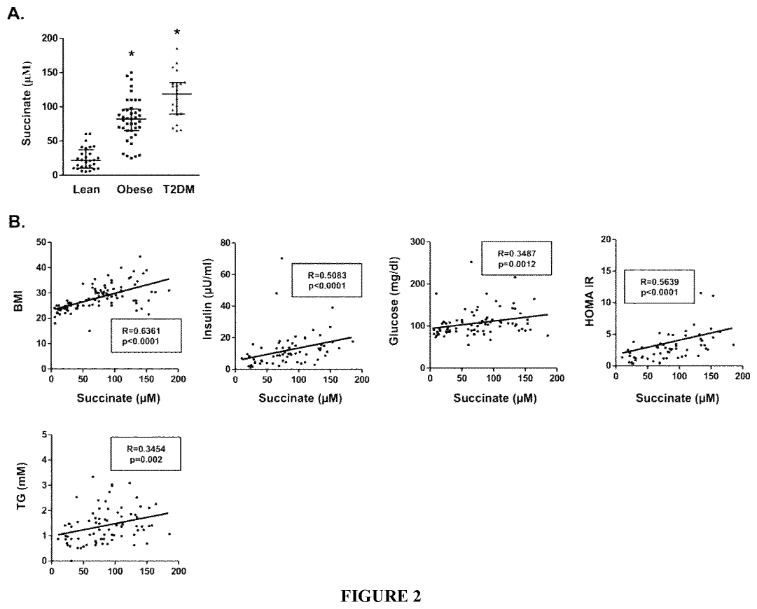

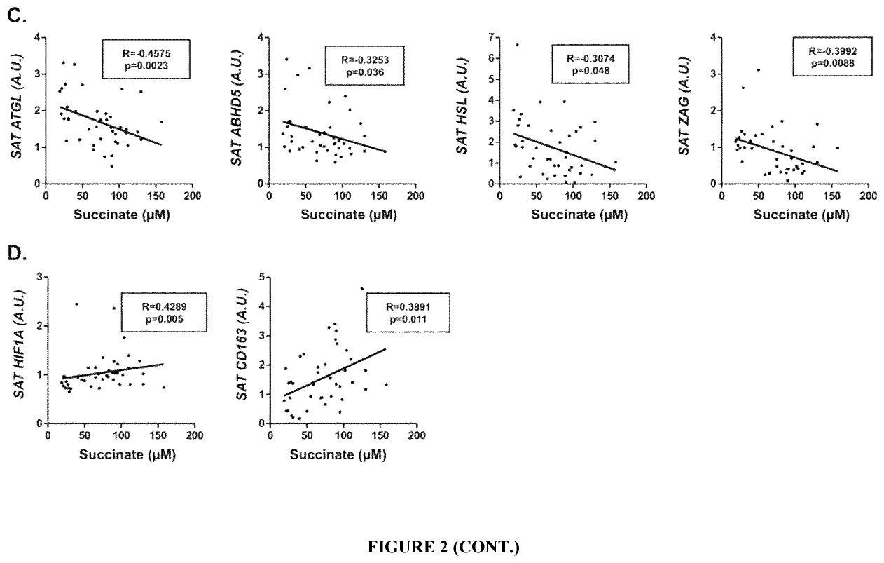

TABLE 2
Anthropometric and analytical characteristics in Cohort II and III studies. Related to FIG. 2.

| | Discovery Cohort II | | | Confirmatory Cohort III | | |
|---|---|---|---|---|---|---|
| | Non-obese | Obese | p | Lean | Obese | p |
| n | 10 | 10 | | 9 | 8 | |
| age | 41.6 ± 4.06 | 43.4 ± 5.56 | ns | 50.22 ± 9.11 | 52.88 ± 7.3 | ns |
| Sex | 10/0 | 10/0 | ns | 4/5 | 6/2 | ns |
| (females/ | | | | | | |
| males) | | | | | | |
| Weight (kg) | 71.1 | 108.7 | 0.001 | 64.8 | 122.25 | 0.001 |
| | (65.2-84.5) | (93.8-121.5) | | (56.05-75.6) | (115.52-125.62) | |
| Waist (cm) | 92.79 ± 5.45 | 124.1 ± 15.39 | <0.001 | 81.89 ± 9.75 | 126.12 ± 14.47 | <0.001 |
| Hip (cm) | 103.9 ± 3.76 | 124.25 ± 15.6 | 0.002 | 98.77 ± 5.87 | 134 ± 14.48 | 0.002 |
| BMI | 25.69 ± 1.6 | 38.82 ± 6.58 | <0.001 | 23.2 | 46.35 | <0.001 |
| | | | | (20.25-24.9) | (38.15-48.07) | |
| Insulin | 8.79 | 19.6 | <0.001 | 2.62 ± 1.92 | 18.4 ± 12 | <0.001 |
| (μU/ml) | (7.99-10.40) | (16.52-33.88) | | | | |
| Glucose | 92 | 112 | <0.001 | 85 ± 5.96 | 99.75 ± 11.2 | <0.001 |
| (mg/dl) | (82.75-93) | (97.75-124.25) | | | | |
| HOMA IR | 1.98 ± 0.28 | 7 ± 3.33 | 0.001 | 0.58 ± 0.44 | 4.33 ± 2.59 | 0.001 |
| SBP | 108.5 | 121 | 0.001 | 121 ± 10.68 | 139 ± 16.96 | 0.001 |
| | (104.5-114.2) | (119.5-136) | | | | |
| DBP | 61.5 | 75.5 | 0.015 | 66.67 ± 11.42 | 78.5 ± 7.43 | 0.015 |
| | (56-71) | (65.75-79) | | | | |
| Cholesterol | 188.6 ± 9.47 | 210.2 ± 43.83 | ns | 200.33 ± 29.71 | 186.75 ± 58.83 | ns |
| (mg/dl) | | | | | | |
| HDL | 65.7 ± 15.50 | 43.6 ± 8.66 | 0.001 | 61.89 ± 17.49 | 52.25 ± 17.5 | 0.001 |
| cholesterol | | | | | | |
| (mg/dl) | | | | | | |
| LDL | 112.86 ± 16.1 | 131.26 ± 31.82 | ns | 72.22 ± 28.97 | 103 ± 65.58 | ns |
| cholesterol | | | | | | |
| (mg/dL) | | | | | | |
| TG (mg/dl) | 85.2 ± 32.04 | 199.2 ± 46.7 | <0.001 | 121 ± 10.68 | 139 ± 16.96 | <0.001 |
| Zonulin | 500.87 ± 44.61 | 869.33 ± 199.01 | 0.03 | 634.48 ± 224.32 | 834.08 ± 315.25 | 0.04 |
| (ng/ml) | | | | | | |

Data are presented as mean ± SD or median (25th-75th), as appropriate. Differences were analyzed by the unpaired t-test (normal distribution) or Mann-Whitney U test (data not-normally distributed). BMI: Body mass index; HOMA-IR: Homeostasis model assessment of insulin resistance index; SBP: Systolic blood pressure; DBP: Diastolic blood pressure; TG: triglycerides. A p value less than 0.05 was considered significant.

Dietary Intervention or Diet Product Cohort IV

Design: Intervention Study.

Participants: Nine obese women (a subsample of the registered study ISRCTN88315555) were included in the study. Subjects were recruited in the outpatient surgery at the Endocrinology Service at the University Hospital Virgen de la Victoria de Milaga. The study participants received no antibiotic treatment, probiotics, prebiotics or any other medical treatment influencing intestinal microbiota during the 3 months before the start of the study.

Intervention: Patients underwent an intervention involving a hypocaloric Mediterranean Diet and physical exercise program. The Mediterranean Diet included extra virgin olive oil and nuts and reduced the energy intake by approximately 600 kcal. The diet comprised fat (35-40%; 8-10% saturated fatty acids), carbohydrates (40-45%; low glycemic index) and protein (20%) (Davis et al., 2015, Nutrients 7:9139-9153; Martinez-Gonzalez and Sanchez-Villegas 2004, Eur. J. Epidemiol. 19:9-13). Adherence to the diet was measured as described previously (Trichopoulou et al., 2003, N. Engl. J. Med. 348:2599-2608). Participants were encouraged to gradually increase their level of physical activity to reach at least 45 minutes per day over the course of the study, which was assessed by their personal trainer on a monthly basis. Participants kept a physical activity record using a GENEActiv© accelerometer. Physical activity levels were evaluated using the Rapid Assessment of Physical Activity questionnaire (Topolski et al., 2006, Prev. Chronic Dis. 3:A118).

Dietary and physical intervention involved individual visits with a nutritionist every week during the 3 months. Furthermore, a nutritional education program was initiated to modify dietary and lifestyle habits with the aim of promoting both weight loss and subsequent weight maintenance. All patients had fasted overnight before collection of blood and stool, before and after the intervention. Anthropometrical and clinical variables are summarized in Table 3. None of the 9 volunteers received antibiotic therapy, prebiotics, probiotics, synbiotics, vitamin supplements or any other medical treatment influencing intestinal microbiota during the 3 months before the start of the study or during the study.

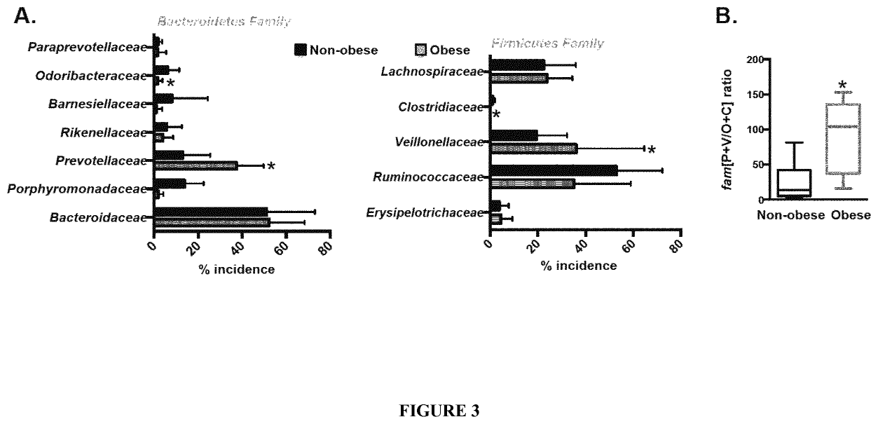

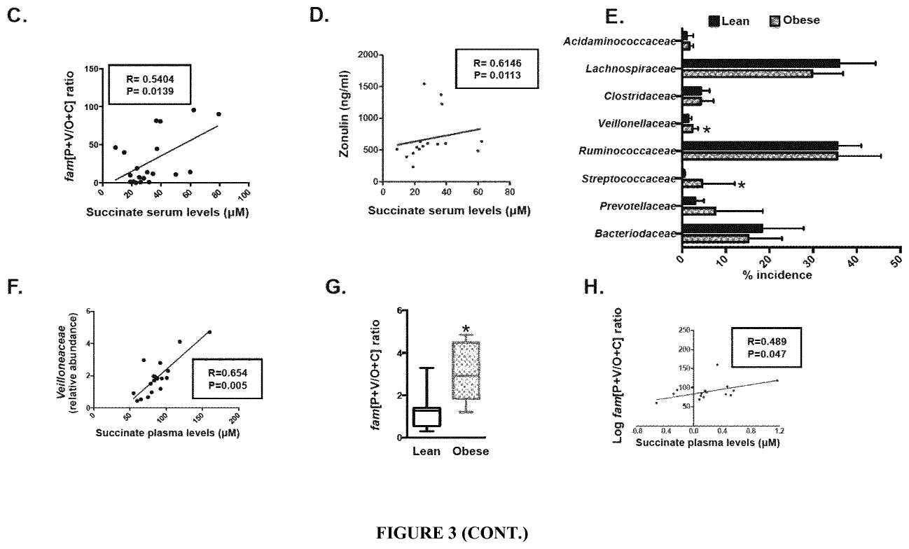

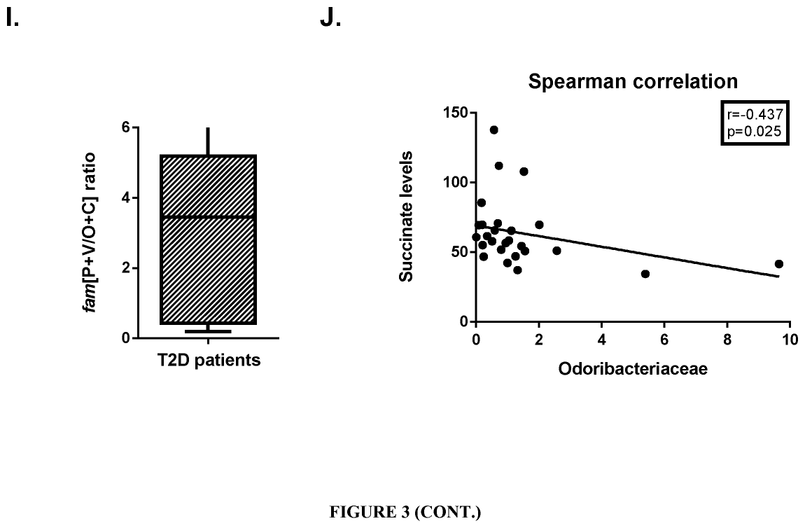

TABLE 3
Anthropometric and analytical characteristics in the dietary intervention
or diet product study cohort IV. Related to FIG. 3.

| | Basal | 12-wDI | p |
|---|---|---|---|
| N | 9 | — | |
| Age (years) | 45.56 ± 4.362 | — | |
| Weight, kg | 93.26 ± 11.83 | 80.12 ± 9.60 | <0.001 |
| BMI, kg/m² | 36.10 ± 4.64 | 31 ± 3.58 | <0.001 |
| Waist (cm) | 115.56 ± 12.32 | 101.56 ± 10.23 | <0.001 |
| Hip (cm) | 121.11 ± 6.80 | 112.22 ± 6.61 | <0.001 |
| Glucose (mM) | 81.78 ± 7.20 | 81.67 ± 7.09 | 0.973 |
| Cholesterol (mM) | 193.89 ± 24.10 | 167.33 ± 26.87 | 0.006 |
| HDL cholesterol (mM) | 57.44 ± 11.57 | 52.22 ± 9.58 | 0.064 |
| LDL cholesterol (mM) | 120.58 ± 18.23 | 101.16 ± 22.43 | 0.013 |
| Triglycerides, mg/dL | 79.33 ± 29.09 | 69.78 ± 15.22 | 0.304 |
| Insulin, μLU/mL | 11.05 ± 4.64 | 9.12 ± 1.39 | 0.393 |
| HOMA-IR | 2.21 ± 0.97 | 1.91 ± 0.28 | 0.444 |
| Hb | 13.20 (12.25-14.10) | 12.90 | 0.514 |
| | | (11.90-13.85) | |
| Hb1ac | 5.17 ± 0.41 | 5.19 ± 0.28 | 0.834 |
| PCR | 4.75 ± 3.07 | 4.41 ± 2.68 | 0.750 |
| TNF | 13.84 ± 1.03 | 13.88 ± 1.11 | 0.864 |
| IL-6 | 4.33 ± 0.47 | 4.54 ± 1.0 | 0.491 |
| Resistin | 4.60 ± 1.51 | 5.21 ± 2.23 | 0.227 |
| Adiponectin | 8.63 ± 2.67 | 6.97 ± 3.12 | 0.142 |
| Succinate (μM) | 57.64 ± 22.23 | 43.06 ± 11.59 | 0.034 |

Data are presented as mean ± SD or median (25th-75th), as appropriate. Differences were analyzed by paired t-test (normal distribution) or Wilcoxon signed-rank test (data not-normally distributed). BMI: Body mass index; HOMA-IR: Homeostasis model assessment of insulin resistance index; SBP: Systolic blood pressure; DBP: Diastolic blood pressure. A p value less than 0.05 was considered significant.

Follow-Up Study Cohort V

Design: Spontaneous Observational Follow-Up Study.

Participants: Nineteen patients were followed for 2 years to evaluate the spontaneous evolution of the microbiota. General counseling was provided to the subjects. None of the 19 volunteers received antibiotic therapy, prebiotics, probiotics, synbiotics, vitamin supplements or any other medical treatment influencing intestinal microbiota in the 3 months before the start of the study or during the study (2 years). All patients had fasted overnight before collection of blood and stool samples, before and after the follow-up period. Anthropometrical and clinical variables are summarized in Table 4.

TABLE 4
Anthropometrical, clinical and microbiota characteristics of
Cohort V. Related to Table 5.
Cohort V

| | |
|---|---|
| n | 19 |
| Sex (females/males) | 10/9 |
| Ratio | 2.20 ± 3.18 |
| Succinate (μM) | 94.22 ± 28.18 |
| Weight (kg) | 77.5 (64.8-121.7) |
| BMI (kg/m²) | 28.5 (23.2-45.8) |
| Waist (cm) | 96 (83-127) |
| Hip (cm) | 107 (101-135) |
| SBP | 128 ± 16.38 |
| DBP | 71.79 ± 10.73 |
| Glucose (mg/dl) | 92.32 ± 10.84 |
| Cholesterol (mg/dl) | 196.32 ± 44.07 |
| HDL cholesterol | 55.63 ± 17.4 |
| (mg/dl) | |
| Tryglycerides (mg/dl) | 85.26 ± 48.12 |
| Hb1ac (%) | 5.59 ± 0.54 |
| Prevotellaceae | 5.39 ± 8.19 |
| Veilloneaceae | 1.8 (0.9-2.3) |
| Odoribacteraceae | ND |
| Clostridaceae | 3.1 (2.8-4.8) |

Data are presented as mean ± SD or median (25th-75th), as appropriate. BMI: Body mass index; SBP: Systolic blood pressure; DBP: Diastolic blood pressure. ND: not detected.

Analytical Determinations

Blood samples were drawn after a 12-h fast. Serum/plasma was separated and immediately frozen at −80° C. Serum biochemical parameters were measured in duplicate. Serum glucose, cholesterol, HDL cholesterol and triglycerides were measured by standard enzymatic methods (Randox Laboratories Ltd., Antrim, UK). Insulin was measured with an immunoradiometric assay (BioSource International, Camarillo, Calif.).

Gene Expression Analysis

Total RNA was extracted from SAT using the RNeasy Lipid Tissue Midi Kit (Qiagen, Hilden, Germany). Total RNA quantity was measured at 260 nm and purity was assessed by the OD260/OD280 ratio. For gene expression analysis, 1 g of RNA was reverse transcribed with random primers using the Reverse Transcription System (Applied Byosistems, Foster City, Calif.). For miRNA analysis, cDNA synthesis was performed with the TaqMan MicroRNA Reverse Transcription Kit (ThermoFisher Scientific, Waltham, Mass.). Real-time PCR (qPCR) was conducted on a 7900HT Fast Real-Time PCR System using TaqMan Gene Expression Assays (Applied Biosystems) for ATGL (Hs 00386101_ml), ZAG (Hs 00426651_ml), ABHD5 (Hs01104373), HSL (Hs 00193510_ml), CD163 (Hs00174705_ml), HIF1A (Hs00153153_ml), ILIB (Hs001749097_ml) and CCL2 (Hs00234140_ml). Results were calculated using the comparative Ct method (2-AACt) and expressed relative to the expression of the housekeeping gene 18 S (Hs 03928985_gi).

Fecal Microbiome Analysis

16S Sequencing (Cohort II and IV)

Collected stool samples were immediately frozen at −80° C. Genomic DNA was extracted following the recommendations of the International Human Microbiome Standards (IHMS; http://www.microbiome-standards.org) (Santiago et al., 2014, BMC Microbiol. 14:112). A frozen aliquot (250 mg) of each sample was suspended in 250 ml of guanidine thiocyanate, 40 ml of 10% N-lauroyl sarcosine, and 500 ml of 5% N-lauroyl sarcosine. DNA was extracted by mechanical disruption of the microbial cells with beads, and nucleic acids were recovered from clear lysates by alcohol precipitation. An equivalent of 1 mg of each sample was used for DNA quantification using a spectrophotometer (NanoDrop Technologies, Wilmington, DL). DNA integrity was examined by micro-capillary electrophoresis using an Agilent 2100 Bioanalyzer with the DNA 12 000 Kit, which resolves the distribution of double-stranded DNA fragments up to 17,000 bp in length. Ribosomal 16S rRNA gene sequences were amplified from cDNA using the 16S Metagenomics Kit (Thermo Fisher Scientific, Italy). The kit included two primer sets that selectively amplify the corresponding hypervariable regions of the 16S region in bacteria: primer set V2-4-8 and primer set V3-6, 7-9. The PCR conditions used were 10 min at 95° C., 30 cycles of 30 s at 95° C., 30 s at 58° C. and 20 s at 72° C., followed by 10 min at 72° C. The concentration and the average size of each amplicon was determined using the Quant-iT PicoGreen dsDNA Assay Kit (Invitrogen); the amount of DNA fragments per microliter was calculated and libraries were created using the Ion Plus Fragment Library Kit (Thermo Fisher Scientific). Barcodes were added to each sample using the Ion Xpress Barcode Adapters 1-16 kit (Thermo Fisher Scientific). The library concentrations were determined using the Ion Universal Library Quantification Kit (Thermo Fisher Scientific). Emulsion PCR and sequencing of the amplicon libraries was performed on a Ion 520 chip (Ion 520™ Chip Kit) using the Ion Torrent S5™ system and the Ion 520™/530™ Kit-Chef (Thermo Fisher Scientific) according to the manufacturer's instructions. After sequencing, the individual sequence reads were filtered using Ion Reporter Software V4.0 to remove low quality and polyclonal sequences.

Metagenomic Analysis (Cohort III and V)

Total DNA was extracted from frozen human stool samples using the QIAamp DNA Stool Mini Kit (Qiagen, Courtaboeuf, France). Quality assessment was performed with the prinseq-lite program applying the following parameters: min_length, 50, trim_qual_right, 20, trim_qual_type, mean; and trim_qual_window, 20. R1 and R2 reads from Illumina sequencing were joined using fastq-join from ea-tools suite. The fastq files were converted into fasta files using the ‘fastq_to_fasta’ tool from the FastX-Toolkit program. Those files were filtered against the human genome, downloaded from the NCBI FTP site (ftp://ftp.ncbi.nlm.nih.gov/genomes/H_sapiens/). The unaligned files, that is, those that did not map against the human genome, were the input files of a BLASTn search against a customized bacterial database (Bacteria 2015_06_09) consisting of the Human Microbiome and the bacterial genomes downloaded from the NCBI FTP site (ftp://ftp.ncbi.nlm.nih.gov/genomes/HUMAN MICROBIOM/Bacteria/and ftp://ftp.ncbi.nlm. nih.gov/genomes/archive/old_refseq/Bacteria/). The best hits of the BLASTn output files were extracted, converted into contingency tables and transformed into BIOM format to be used as input files of the Quantitative Insights Into Microbial Ecology (QIIME) open-source software pipeline version 1.9.0 (Langmead and Salzberg, 2012, 9:357-359; Schmieder and Edwards, 2011, Bioinformatics 27:863-864).

Circulating Succinate Measurement

Fluorimetric Method

Circulating serum/plasma succinate levels were measured using the EnzyChrom™ Succinate Assay Kit (BioAssay Systems, Hayward, Calif.). The assay sensitivity was 12 μM and the intra- and interassay co-efficients of variance were less than 3.5 and 6.95%, respectively.

LC-MS/MS and NMR Analysis

Circulating succinate levels obtained by the fluorimetric assay were validated using LC-MS/MS and NMR analysis. To do this, a sub-sample of plasma samples from cohort I was prepared as previously reported with some modifications (Nagana Gowda et al., 2015, Anal. Chem. 87:706-715; Tulipani et al., 2013, Anal. Chem. 85:341-348). Importantly, the concentration of succinic acid measured by the fluorimetric assay correlated with that measured by LC-MS/MS (r=0.617, p=0.019) and by NMR (r=0.769, p=0.043), indicating that the fluorimetric assay could be used to measure human succinate levels, which is faster and more economical than the other two methodologies.

Circulating Zonulin Measurement

Serum zonulin was measured as a surrogate marker of intestinal permeability. Circulating plasma/serum zonulin levels were assessed using the Human Zonulin Elisa Kit (MyBiosource, San Diego, Calif.) (Smecuol et al., 2005, Clin. Gastroenterol. Hepatol. 3:335-341; Wang et al., 2000, J. Cell Sci. 113 Pt 24:4435-4440). This assay has high sensitivity (1 ng/ml) and excellent specificity for detection of zonulin, and only detects the active (uncleaved) form. The intra- and interassay coefficients of variation for these determinations were <10%.

Statistical Analysis

Statistical analysis was performed with the Statistical Package for the Social Sciences software, version 15 (SPSS, Chicago, Ill.). For clinical and anthropometrical variables, normally distributed data were expressed as mean±SD and for variables with no Gaussian distribution values were expressed as median (25th-75th quartiles). Student's t test with Bonferroni adjustment was used to compare the mean value of normally distributed continuous variables. For variables that did not have a Gaussian distribution, the Kruskal-Wallis test was used with post hoc Dunn's multiple comparison test. To analyze the differences in nominal variables between groups, the χ² test was used. For microbiota data, statistical significance was tested by unpaired t-test or Mann-Whitney U test as part of the SPSS software package. For intervention studies, Wilcoxon signed-rank test or paired t-test was used for paired analysis in the two prospective cohorts as appropriate. Pearson's and Spearman's correlation coefficients with Bonferroni adjustment were used to analyze the relationship between parameters. To determine which variables were associated with circulating succinate, multiple linear regression analyses were employed (stepwise forward selection procedures). All variables associated in the univariate analysis with succinate were included in their respective models. A p value less than 0.05 was considered significant. For functional studies, statistical analysis was performed with R statistics software version 3.3.3. Wilcoxon rank-sum test was used for hypotheses testing analysis between the two groups (group 1 vs. group 2). Heat maps were generated using a hierarchical clustering algorithm to visualize the metagenomic function and metabolite differences within the dataset.

Succinate Threshold Level Associated with an Altered Metabolic Profile

An altered metabolic profile in a subject is defined as a set of threshold values for a number of parameters which are associated with the risk of developing metabolic pathologies such as diabetes. The values characteristic of an altered metabolic profile are as follows:
Insulin >25 μLU/mL
Glucose >100 (mg/dl)
HOMA-IR >3.21
Triglycerides >1.7 (mM)

The threshold values for glucose and triglycerides are values defined by the American Diabetes Association, American Heart Association or by the International Diabetes Federation in order to define the metabolic syndrome. However, in the context of the invention these thresholds do not necessarily relate to Metabolic Syndrome. The threshold value for HOMA-IR (Homeostasis model assessment of insulin resistance index) has been described elsewhere (Ceperuelo-Mallafré et al., J Clin Endocrinol Metab. 2014 May; 99(5):E908-19; Cardona F. et al., Clin Chem. 2006 October; 52(10):1920-5).

Based on the data from the 94 patients from Cohort I (Table 1), the inventors have calculated the threshold value for circulating succinate associated with an altered metabolic profile as defined above. In particular, the inventors have used the CART (Classification and Regression Tree) statistical method to determine the succinate values characteristic of subjects with an “altered” or subjects with an “optimal” metabolic profile. The CART method was performed with the Statistical Package for the Social Sciences software, version 19 (SPSS, Chicago, Ill.). The CART method is a graphic representation of a series of decision rules. CART is a stepwise, nonparametric procedure in which the classification potential of variables is assessed relative to a split. Subjects with values less than the cutoff point move to one category, whereas those with values greater than the cutoff point move into a second box of the tree. The main elements of CART are: (a) rules for splitting data at a node based on the value of one variable; (b) stopping rules for deciding when a branch is terminal and can be split no more; and (c) finally, a prediction for the target variable in each terminal node. The circulating succinate threshold value obtained with this method for blood samples is 60.390 μM (FIG. 1A), whereas the circulating succinate threshold level for urine samples is 10.250 μM (FIG. 1B).

### Example 1: Circulating Levels of Succinate are Elevated in Obesity and Associate with a Worse Metabolic Profile

In a cohort of 91 patients stratified according to obesity and T2DM (cohort 1), plasma succinate levels were significantly higher in obese than in lean individuals (FIG. 2A, Table 1), and a comparable increase was detected in BMI-matched T2DM patients, in line with a recent report (van Diepen et al., 2017, Diabetologia 60:1304-1313). These results suggest that systemic succinate is also associated with body weight status. Accordingly, a positive association was found between circulating succinate levels and BMI (FIG. 2B), but also insulin, glucose, homeostasis model assessment of insulin resistance (HOMA-IR) and triglycerides (FIG. 2B). Consistent with the documented role of succinate in blood pressure regulation (He et al., 2004, Nature 429:188-193; Sadagopan et al., 2007, Am. J. Hypertens. 20:1209-1215), circulating succinate also correlated positively with diastolic blood pressure (R=0.386, p=0.039). A multiple regression analysis model (R2=0.295) adjusted for age and gender showed that BMI and glucose (β=0.495 p<0.001 and β=0.279 p=0.013, respectively) were the main determinants of circulating succinate levels.

Succinate has been shown to have antilipolytic actions in adipose tissue via engagement with SUCNR1, inhibiting the release of fatty acids from adipocytes (McCreath et al., 2015, Diabetes 64:1154-1167; Regard et al., 2008, Cell 135:561-571). Consistent with this scenario, metabolic gene expression profiling in SAT from a representative subset of cohort I (n=42) revealed a negative association between systemic succinate levels and genes encoding key enzymes involved in intracellular degradation of triacylglycerols, including adipose triglyceride lipase (ATGL), abhydrolase domain containing (ABHD5) and hormone-sensitive lipase (HSL) (FIG. 2C. A similar negative association was found for the gene encoding the secreted AT lipolytic factor zinc-alpha-2-glycoprotein (ZAG) (FIG. 2C). Conversely, a positive association was found between succinate and hypoxia-inducible factor HIF-1α (FIG. 2D), a key transcription factor underlying chronic inflammation and AT dysfunction in obesity (Trayhurn et al., 2008, Am. J. Physiol. Regul. Integr. Comp. Physiol. 295:R1097; Ye, 2009, Int. J. Obes. (Lond.) 33:54-66). Indeed, a clear function for succinate has been established in innate immune signaling, where it enhances interleukin-1 beta (IL-1β) production via stabilization of HIF-1α (Corcoran and O'Neill 2016, J. Clin. Invest. 126:3699-3707; Tannahill et al., 2013, Nature 496:238-242). Nevertheless, systemic succinate levels were found to be associated with the expression of the anti-inflammatory macrophage marker CD163 in SAT (FIG. 2D), but not with inflammatory markers such as IL-10 or MCP-1 (R=0.116 p=0.466; R=0.039 p=0.809, respectively), supporting the notion that succinate might have differential intracellular and extracellular functions as previously noted for other stress-related factors such as osteopontin and heat shock proteins. Of note, while some associations were also found in visceral adipose tissue, stronger correlations were detected in SAT, suggesting that the subcutaneous fat depot is more responsive to succinate than visceral fat.

### Example 2: Gut Microbiota Composition is Associated with Circulating Succinate Levels

In an independent cohort (cohort II, clinical and anthropometrical characteristics are summarized in Table 2), the serum concentration of succinate was significantly higher in obese than in non-obese individuals (43.936.16 μM vs. 23.2±1.57 μM, p=0.0020). Of note, the concentration of succinate in serum is about one-third lower than that found in plasma (Ariza et al., 2012, Front. Endocrinol. (Lausanne) 3:22, and this study).

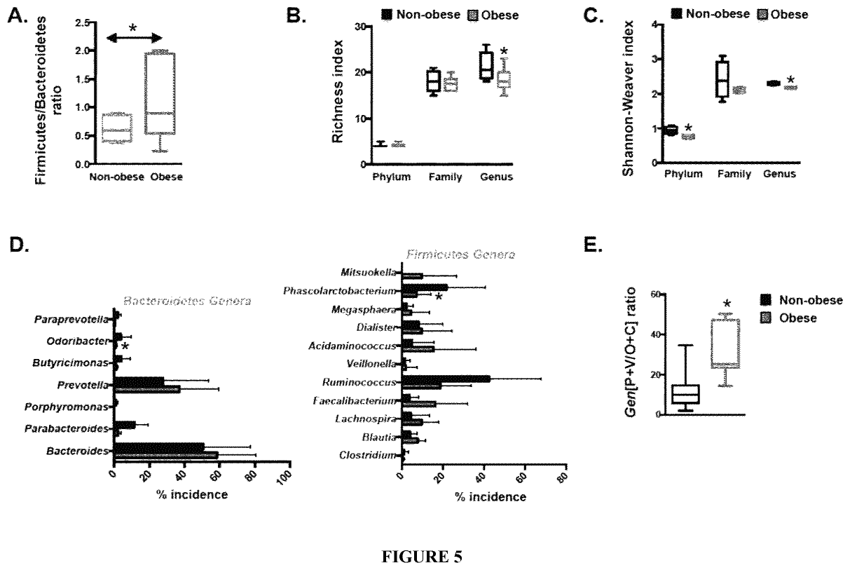

Analysis of gut microbiota composition by 16S rRNA gene sequencing revealed an increase in the Firmicutes/Bacteroidetes ratio in obese subjects (FIG. 3A), and decreased richness and biodiversity at the phylum and genus level (FIG. 5B-C) (Duncan et al., 2008, Int. J. Obes. (Lond.) 32:1720-1724; Ley et al., 2005, Proc. Natl. Acad. Sci. USA 102:11070-11075; Ley et al., 2006, Nature 444:1022-1023; Zhang et al., 2009, Proc. Natl. Acad. Sci. USA 106:2365-2370). The relative abundance (RA) of Prevotellaceae (37.52±3.86% vs. 12.93±3.97%, p=0.0005) and Veillonellaceae (36.08±9.52% vs. 19.51±4.26%, p=0.03), known succinate producers (Louis et al., 2014, Nat. Rev. Microbiol 12:661-672; Nakayama et al., 2017, Front. Microbiol. 8:197; Vogt et al., 2015, Anaerobe 34:106-115), was found to be higher in obese than in non-obese individuals (FIG. 3A). Accordingly, serum succinate levels positively correlated with Prevotellaceae (R=0.465; p=0.039). Conversely, the RA of Odoribacteraceae (1.58±0.68% vs. 6.18±1.64%, p=0.005) and Clostridaceae (0.09±0.04% vs. 1.02±0.36%, p=0.05) families, known succinate consumers (Ferreyra et al., 2014, Cell Host Microbe. 16:770-777; Reichardt et al., 2014, ISME J. 8:1323-1335), was significantly lower in obese than in non-obese individuals (FIG. 3A). No differences were detected in other bacterial families such as Paraprevotellaceae, Bacteroidaceae or Ruminococcaceae, which are also related to succinate metabolism (Ferreyra et al., 2014, Cell Host Microbe 16:770-777; Louis et al., 2014, Nat. Rev. Microbiol 12:661-672; Morotomi et al., 2008, Int. J. Syst. Evol. Microbiol. 58:2716-2720; O'Herrin and Kenealy 1993, Appl. Environ. Microbiol. 59:748-755; Watanabe et al., 2012, Appl. Environ. Microbiol. 78:511-518). Consequently, the ratio of [(Prevotellaceae+Veillonellaceae)/(Odoribacteraceae+Clostridaceae)] (fam[P+V/O+C]), specific succinate producers per consumers, was significantly higher in obese subjects (FIG. 3B) and correlated positively with succinate serum levels (FIG. 3C). At the genus level, the succinate-producing member Mitsuokella spp. was found to be enriched in fecal samples of obese subjects (9.67±5.37% vs. 0.11±0.11%, p=0.08), which was accompanied by a significant decrease in the succinate-consuming members Phascolarctobacterium spp. (7.27±2.29% vs. 24.15±6.12%, p=0.018) and Odoribacter spp. (0.8±0.27% vs. 3.66±1.81%, p=0.017) (FIG. 5D). Correspondingly, the ratio of specific succinate-producers/succinate-consumers at the genus level was also significantly higher in obese than in non-obese individuals (FIG. 5E).

According to the “leaky gut” hypothesis, intestinal dysbiosis characteristic of obesity is directly related to translocation of bacteria and their products into systemic circulation (Slyepchenko et al., 2016, Curr. Pharm. Des. 22:6087-6106). As expected, circulating levels of zonulin, a useful biomarker of intestinal permeability, were significantly higher in obese than in non-obese individuals (869.33199.013 ng/ml vs. 500.8744.61 ng/ml, p=0.04). A positive correlation was found between serum succinate and circulating zonulin (R=0.61; p=0.011) (FIG. 3D), suggesting that akin to the elevated levels of circulating lipopolysaccharide in obesity, intestinal permeability might be closely associated with the presence of succinate in systemic circulation.

To further investigate the relationship between serum succinate and the gut-microbiome, a whole-genome shotgun sequencing of fecal DNA was performed in an independent cohort (confirmatory cohort III; clinical and anthropometrical characteristics summarized in Table 2). As noted in previous cohorts, succinate plasma levels were significantly higher in obese than in lean individuals (101.72±9.37 μM vs. 78.24±4.4 μM, p=0.043). Furthermore, a significant increase in the family Veillonellaceae (2.37±0.39% vs. 1.41±0.24%, p=0.043) was found in obese subjects (FIG. 3E), as well as a positive correlation between Veillonellaceae and succinate levels in plasma (R=0.773; p<0.001) (FIG. 3F). Accordingly, obese subjects had a higher fam[(P+V)/(O+C)] ratio (FIG. 3G), which positively correlated with plasma succinate levels (FIG. 3H). Similar to cohort II, obese individuals had higher zonulin levels (Table 2), which also positively associated with circulating succinate levels (R=0.59; p=0.0152). An even higher fam[(P+V)/(O+C)] ratio was found for obese, diabetic subjects (FIG. 3I). In this sub-group of patients, a correlation was found between Odoribacteriaceae and succinate levels in plasma (FIG. 3J).

Overall, these data demonstrate that despite the interindividual heterogeneity, circulating succinate levels are associated with specific components of gut microbiota. Interestingly, the microorganisms linked to circulating succinate levels have been previously related to CVD and/or its risk factors. Thus, succinate-consuming genera such as Odoribacter and Clostridium have been linked to a decrease in clinical parameters associated with CVD risk (Karlsson et al., 2012, Nat. Commun. 3:1245; Tang et al., 2017, Circ. Res. 120:1183-1196). By contrast, the Prevotella genus, which was found to be increased in obese individuals, has been recently associated with hypertension (Li et al., 2017b, Microbiome 5:14) and TMAO-induced atherosclerosis (Koeth et al., 2013, Nat. Med. 19:576-585; Org et al. 2015, Atherosclerosis 241:387-399). Along these lines, Chen and colleagues have demonstrated that resveratrol modulates gut microbiota by inhibiting the Prevotella genus, which in turn induces a decrease in circulating TMAO levels (Chen et al., 2016, MBio 7:e02210-02215), pointing to gut microbiota as an attractive target for pharmacological or dietary intervention or diet products to decrease the risk of developing CVD.

### Example 3: Modification of Gut Microbiota by Dietary-Weight Loss Intervention Affects Circulating Succinate Levels

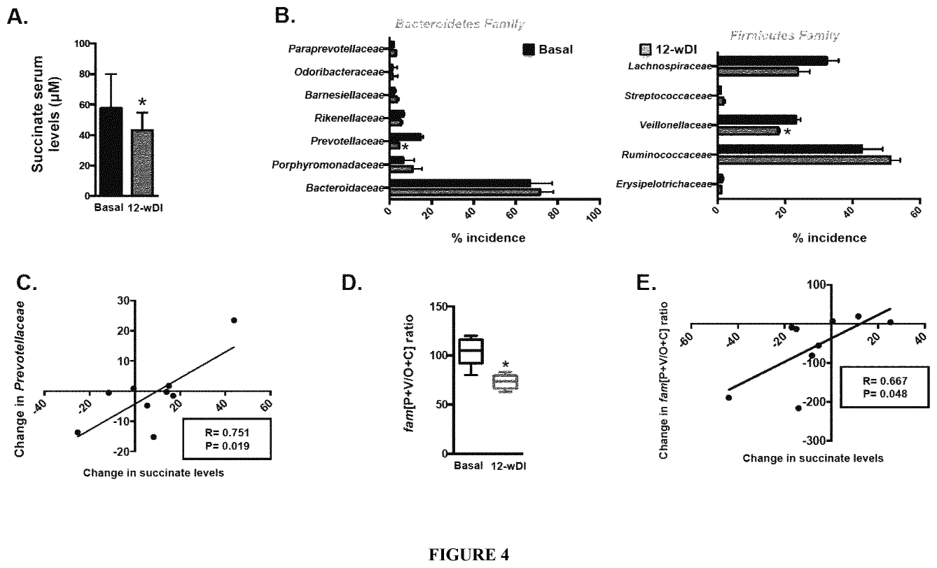

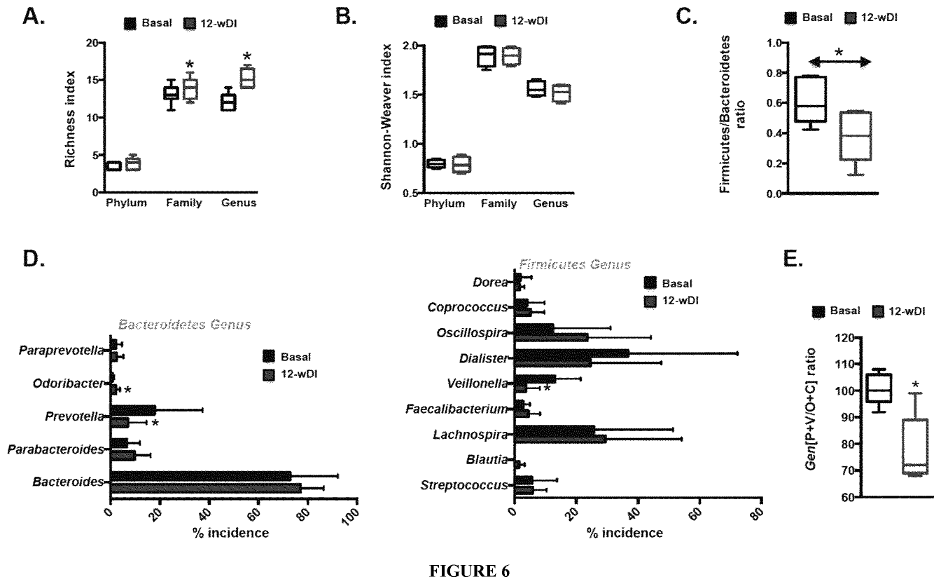

To determine whether diet-induced modifications in gut microbiota could be reflected in variations in circulating succinate levels, a prospective 12-week dietary intervention or diet product study was carried out in obese patients aimed to weight loss (cohort IV, Table 3). Serum succinate levels decreased after the intervention (FIG. 4A) in parallel with an increase in genus and family richness (FIG. 6A). While no significant differences were detected in genus or family diversity (FIG. 6B), a decrease in the Firmicutes/Bacteroidetes ratio (FIG. 6C) was identified, similar to that reported in a previous dietary-weight loss intervention study (Cotillard et al., 2013, Nature 500:585-588; Dao et al., 2016, Clinical Nutrition Experimental 6:39-58; Healey et al., 2017, Nutr. Rev. 75:1059-1080).

In accordance with the results of the two previous cohorts (cohort II and III), a significant decrease in the succinate-producing families Prevotellaceae (17.916.43% vs. 7.15±2.47%, p=0.019) and Veillonellaceae (13.11±2.76% vs. 3.73±1.48%, p=0.027) was found after the dietary intervention or diet product (FIG. 4B). Comparable to that observed in cohort III, a positive correlation was found between the change in the incidence of Prevotellaceae ([Prevotellaceae]ₚₒₛₜ₋ᵢₙₜₑᵣᵥₑₙₜᵢₒₙ−[Prevotellaceae]_{basal}) and succinate levels (R=0.751; p=0.019) (FIG. 4C). Correspondingly, the fam[(P+V)/(O+C)] ratio significantly decreased after weight loss (FIG. 4D) in parallel with a decrease in succinate, which was reflected in a positive correlation between the change in the fam[(P+V)/(O+C)] ratio and the change in circulating succinate (post-intervention−basal) (FIG. 4E). Similar observations were found at the genus level (FIG. 6D), and the gen[(P+V)/(O+C)] ratio significantly decreased after the intervention (FIG. 6E).

Taken together, these results indicate that a short-term dietary weight loss intervention impacts different members of the gut commensal community related to succinate metabolism. Specifically, a decrease in succinate-producers concomitant with an increase in succinate consumers at two taxonomic levels, which correlates with the decrease in systemic succinate levels observed, pointing to circulating succinate as a new dysbiosis-associated metabolite in the context of obesity.

Remarkably, joint analysis of both microbiota cohorts (cohort II and IV) validated the strong positive correlation between the fam[(P+V)/(O+C)] ratio and circulating serum succinate levels (n=38, R=0.646; p<0.001). Reassuringly, multiple regression analysis revealed that our proposed ratio based on [succinate-producing] vs. [succinate-consuming] families was the main determinant of systemic succinate levels (R²=0.744, β=0.597; p=0.007). Notwithstanding these strong correlations, exactly how microbial communities interact and use succinate is currently unknown. Moreover, other microbial groups could be responsible for succinate production (e.g., Succinovibrio spp., Ruminococcus spp. or Fibrobacter succinogenes) and consumption (e.g., Dialister spp., Phascolartobacterium succinatutes) (Ferreyra et al., 2014, Cell Host Microbe. 16:770-777; Louis et al., 2014, Nat. Rev. Microbiol 12:661-672; Morotomi et al., 2008, Int. J. Syst. Evol. Microbiol. 58:2716-2720; O'Herrin and Kenealy 1993, Appl. Environ. Microbiol. 59:748-755; Watanabe et al., 2012, Appl. Environ. Microbiol. 78:511-518). Nevertheless, our results strongly link the specific fam[(P+V)/(O+C)] ratio to circulating succinate.

### Example 4: Microbiota Spontaneous Evolution Drives Changes in Systemic Succinate

Finally, to evaluate the spontaneous evolution of microbiota, 19 subjects in whom general healthy habits counseling was provided were studied: at baseline and at 2 years thereafter (see Methods section, cohort V description in Table 4). No significant differences in body weight were observed in these patients in the follow-up. A metagenomic approach was used rather than 16S sequencing to analyze gut microbiota in this cohort. At the end of follow-up, subjects were classified into two groups in terms of changes in the ratio [succinate-producing] vs. [succinate-consuming] families (group 1, decreased ratio vs. group 2, increased ratio). Areduction infam[(P+V)(O+C)] was associated with significant decrease in succinatelevels (Table 5, group 1), whereasa significant increase in this ratio was related to arise in systemic succinate (Table 5, group 2).

TABLE 5
Anthropometric and analytical characteristics in the cohort V.
Follow-up study (cohort V)

| | Group 1 | Group 2 | p |
|---|---|---|---|
| ΔRatio P + V/C + O | −1.14 ± 1.29 | 2.83 ± 3.20 | 0.002 |
| n | 8 | 11 | |
| Sex (females/males) | 6/2 | 4/7 | 0.096 |
| ΔSuccinate (mM) | −25.19 ± 27.11 | 12.33 ± 32.75 | 0.017 |
| ΔWeight (kg) | −0.25 (−19.1-3.47) | 1.8 (−1-3.8) | 0.351 |
| ΔBMI (kg/m²) | 0.15 (−3.35-1.42) | 0.30 (−0.4-1.3) | 0.840 |
| ΔWaist (cm) | 8 (−39-11) | 8 (4.75-10.5) | 0.475 |
| ΔHip (cm) | 2 (−33-6) | 3 (−0.5-3.75) | 0.887 |
| ΔSBP | −0.71 ± 23.73 | 3.20 ± 16.56 | 0.693 |
| ΔDBP | −1.71 ± 13.74 | 6.8 ± 11.50 | 0.185 |
| ΔGlucose (mg/dl) | −1 ± 7.78 | 6 ± 6.15 | 0.042 |
| ΔCholesterol (mg/dl) | 6.12 ± 30.60 | −4.09 ± 22.83 | 0.415 |
| ΔHDL cholesterol | 2.87 ± 12.91 | 0.55 ± 7.12 | 0.620 |
| (mg/dl) | | | |
| ΔTriglycerides (mg/dl) | 1.61 ± 21.61 | 6.63 ± 27.56 | 0.675 |
| ΔHb1ac (%) | 0.41 ± 0.70 | 0.4 ± 0.68 | 0.969 |
| ΔPrevotellaceae | −3.66 ± 4.29 | 6.21 ± 7.68 | 0.005 |
| ΔVeillonellaceae | −0.44 ± 1.89 | −0.7 ± 1.38 | 0.728 |
| ΔOdoribacteraceae | ND | ND | — |
| ΔClostridaceae | 0.26 (−0.66-0.44) | −1.1 (−2.82-−0.58) | 0.039 |

Data are presented as mean ± SD or median (25th-75th), as appropriate. Differences were analyzed by the unpaired t-test (normal distribution) or Mann-Whitney U test (data not-normally distributed). Group 1 (patients ratio decreases at the end of follow-up) and Group 2 (patients ratio increases at the end of follow-up); BMI: Body mass index; SBP: Systolic blood pressure; DBP: Diastolic blood pressure. ND: not detected. A p value less than 0.05 was considered significant.

These results demonstrate that variation in gut microbial composition independent of body weight changes are directly related to circulating succinate. Of note, elevated systemic succinate was paralleled with an impairment of glucose homeostasis, which contrasts with recently reported findings in animal models showing that microbiota-produced succinate is directly related to an improvement of glucose homeostasis (De Vadder et al., 2016, Cell Metab. 24:151-157). Indeed, high succinate levels have been associated with various human pathological settings including cardiovascular disease (Aguiar et al., 2014, Cell Commun. Signal. 12:78) and T2DM (Guo et al., 2017, Nat. Commun. 8:15621; Sadagopan et al., 2007, Am. J. Hypertens. 20:1209-1215; Toma et al., 2008, J. Clin. Invest. 118:2526-2534; van Diepen et al., 2017, Diabetologia 60:1304-1313).

Multivariate analyses identified statistically significant associations between the expression of 64 genes encoding metabolic enzymes, and the fam[(P+V)/(O+C)] ratio. Hierarchical clustering of these metagenomic data and the associations among fam[(P+V)/(O+C)] ratio, circulating succinate and succinate-related microbial species, identified two clusters (labeled A and B, data not shown) with a clear relationship with the fam[(P+V)/(O+C)] ratio, which was mostly reflected by succinate levels. The metagenomic-derived clusters were also confirmed when associations with Prevotellaceae and Clostridaceae were analyzed, and a strong inverse relationship was detected. The main positive associations in cluster A were with genes encoding metabolic enzymes involved in amino acid transport and metabolism ([E]), whereas cluster B showed a predominance of associations with genes related to energy production and conversion ([C]). Robust relationships with genes related to carbohydrate transport and metabolism ([G]) were revealed in both clusters. Interestingly, sub-clusters A1/A2 and B1/B2 were segregated on the basis of inverse associations with Veillonellaceae and Clostridaceae. These results link the fam[(P+V)/(O+C)] ratio, specific-gut microbiota and circulating succinate levels with a specific molecular entity and metabolic function.

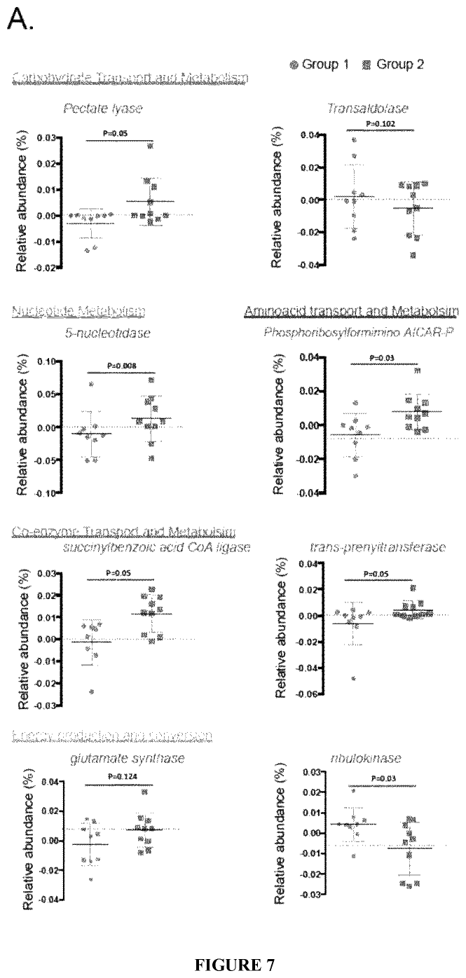

The differences in the gene expression profiles associated with specific bacterial communities were also evident when the cohort was classified into two groups according thefam[(P+V)/(O+C)] ratio (group 1 vs. group 2) (FIG. 7A). An increase in the abundance of genes encoding enzymes associated with carbohydrate transport and metabolism ([G]), such as pectate lyase [EC:4.2.2.2], pectinesterase [EC:3.1.1.11] and glycosyl hydrolase [EC:3.2.1.52] after 2 years of follow-up, was detected in subjects in whom the fam[(P+V)/(O+C)] ratio was increased in parallel with an increase in succinate levels. Curiously, a decrease in the abundance of genes encoding enzymes connecting the pentose phosphate pathway to glycolysis, such as ribulokinase [EC:2.7.1.16] and transaldolase [EC:2.2.1.2], was also observed in these patients. Genes associated with metabolic pathways linked to the biosynthesis of secondary metabolites ([Q]) such as succinylbenzoic acid-CoA ligase [EC:6.2.1.26], or those associated with amino acid transport and metabolism ([E]) such as phosphoribosylformimino-5-aminoimidazole carboxamide ribotide isomerase [EC:5.3.1.16] and glutamate synthase [EC:1.4.1.14] were also modified. Intriguingly, all of these genes showed the strongest association with the fam[(P+V)/(O+C)] ratio (data not shown). More importantly, projection of these enzymes onto the KEGG metabolic pathways map identified central metabolism as the main process associated with the fam[(P+V)/(O+C)] ratio. Among them, glyocoside hydrolase and glutamate synthase were of particular interest because of their functional roles in glycolysis activation and succinate production via the GABA shunt pathway. Also worthy of mention was the negative association of fam[(P+V)/(O+C)] ratio with ribulokinase and transaldolase, which also could promote glycolysis through inhibition of the pentose phosphate pathway (data not shown). Mapping of the main enzymes positively or negatively correlated with fam[(P+V)/(O+C)] ratio uncovered a clear connection between their functional features and succinate metabolism (adapted from KEGG metabolic pathways) (data not shown).

In conclusion, the present study reveals for the first time a strong association between microbial community, gene composition and metabolism and circulating succinate levels in humans.

### Example 5: Glucose Tolerance Test in Obese Mice Treated with Odoribacter laneus

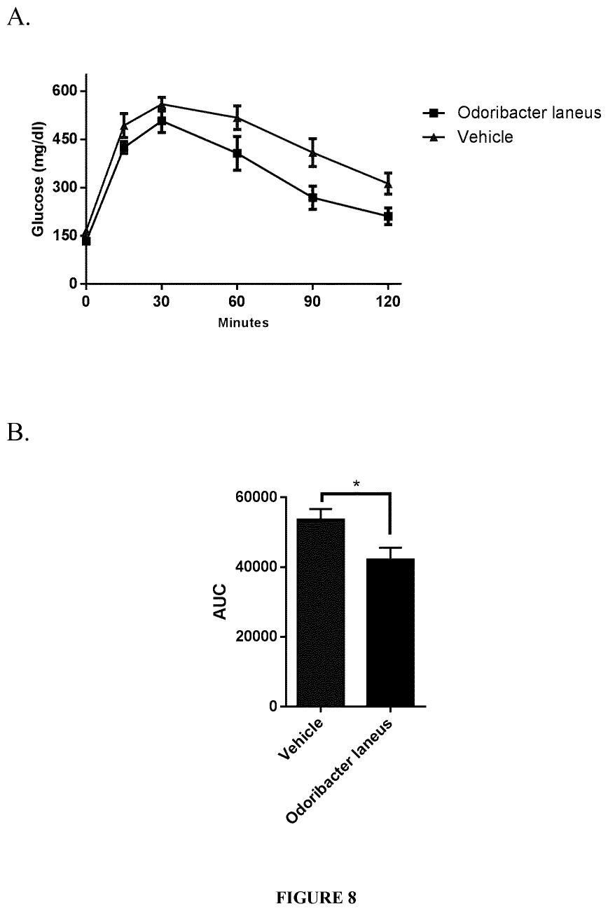

C57/B16 mice were fed with a High Fructose Diet for 16 weeks. Obese mice were then daily treated with Odoribacter laneus with 100 uL bacteria at 1×10⁹ CFU/mL in PBS+glycerol 1% (vehicle) with an oral gavage for 24 days. Glucose Tolerance (FIG. 8A) improved in Odoribacter laneus-treated animals. The area under the curve (AUC) is shown in (FIG. 8B).

1-24. (canceled)

25. A kit comprising reagents suitable for determining the ratio of succinate-producing bacteria to succinate-consuming bacteria in a stool sample from a subject,
wherein the kit comprises primer sets designed to specifically hybridize the hypervariable regions of the 16S rRNA gene in at least one succinate-producing bacterium and in at least one succinate-consuming bacterium, or
wherein the kit comprises probes that specifically hybridize to the hypervariable regions of the 16S rRNA gene in at least one succinate-producing bacterium and in at least one succinate-consuming bacterium,
and wherein the primer sets or the probes comprise at least 10% of the total amount of reagents forming the kit.

26. The kit according to claim 25 wherein the ratio of succinate-producing bacteria to succinate-consuming bacteria is the (Prevotellaceae+Veillonellaceae)/(Odoribacteriaceae+Clostridiaceae) ratio.

27. A method selected from the group consisting of:
(A) a method to detect the ratio of succinate-producing bacteria to succinate-consuming bacteria in a stool sample from a subject comprising using the kit according to claim 25;
(B) a method to determine whether the succinate level in a biofluid sample from a subject is above a threshold level, comprising using a kit, said kit comprising reagents suitable for determining the succinate level in a biofluid sample from a subject
wherein the presence of succinate in said biofluid sample above a predetermined threshold level provides a positive result and
wherein the presence of succinate in said biofluid sample below a predetermined threshold level or the absence of succinate in said biofluid sample provides a negative result;
(C) a method to determine whether a probiotic intervention directed at reducing the levels of circulating succinate in a subject has been effective comprising using a kit, said kit comprising reagents suitable for determining the succinate level in a biofluid sample from a subject, wherein
a level of circulating succinate in the biofluid sample from the subject after the probiotic intervention lower than the level of circulating succinate in the biofluid sample from the subject before the probiotic intervention is indicative that the probiotic intervention has been effective,
and wherein
a level of circulating succinate in the biofluid sample from the subject after the probiotic intervention equal to or higher than the level of circulating succinate in the biofluid sample from the subject before the probiotic intervention is indicative that the probiotic intervention has not been effective; and
(D) a method for determining whether a targeted intervention directed at reducing the levels of circulating succinate in a subject has been effective, the method comprising:
(a) determining the ratio of succinate-producing bacteria to succinate-consuming bacteria in a stool sample from the subject before the targeted intervention, and
(b) determining the ratio of succinate-producing bacteria to succinate-consuming bacteria in a stool sample from the subject after the targeted intervention,
wherein
a ratio of succinate-producing bacteria to succinate-consuming bacteria in the stool sample from the subject after the targeted intervention lower than the ratio of succinate-producing bacteria to succinate-consuming bacteria in the stool sample from the subject before the targeted intervention is indicative that the targeted intervention has been effective,
and wherein
a ratio of succinate-producing bacteria to succinate-consuming bacteria in the stool sample from the subject after the targeted intervention equal to or higher than the ratio of succinate-producing bacteria to succinate-consuming bacteria in the stool sample from the subject before the targeted intervention is indicative that the targeted intervention has not been effective.

28. The method according to claim 27(A), wherein the ratio of succinate-producing bacteria to succinate-consuming bacteria is the (Prevotellaceae+Veillonellaceae)/(Odoribacteriaceae+Clostridiaceae) ratio.

29. The method according to claim 27(B), wherein the biofluid sample from the subject is a blood sample, a urine sample or a stool sample.

30. The method according to claim 29, wherein if the biofluid is blood the succinate threshold level is between 50 and 70 μM, or if the biofluid is urine the succinate threshold level is between 5 and 15 μM.

31. The method of claim 27(D), wherein the targeted intervention is selected from the group consisting of a dietary intervention or diet product, a pharmacological intervention, and a probiotic intervention.

32. The method according to claim 27(D), wherein the patient is obese.

33. The method according to claim 27(D), wherein the patient has type 2 diabetes mellitus.

34. A method for the treatment and/or prevention of a disease associated with increased levels of circulating succinate in a patient selected from the group consisting of:
a) a method comprising subjecting the patient to a dietary intervention or providing the patient with a diet product, wherein the intervention decreases the ratio of succinate-producing bacteria to succinate-consuming bacteria in the intestinal tract of the patient;
b) a method comprising administering a pharmacological product or a probiotic product to the patient, wherein the product decreases the ratio of succinate-producing bacteria to succinate-consuming bacteria in the intestinal tract of the patient; and
c) a method comprising administering a pharmacological product or a probiotic product to the patient, wherein the product decreases the levels of circulating succinate of the patient.

35. The method according to claim 34, wherein the intervention comprises a hypocaloric diet characterized in that:
fat is 35-40% of total daily calorie intake; and
carbohydrates are 40-45% of total daily calorie intake;
wherein the dietary intervention or diet product is administered for at least 12 weeks, and
wherein the dietary intervention or diet product is optionally administered in combination with a physical exercise program.

36. The method according to claim 34, wherein the patient is obese.

37. The method according to claim 34, wherein the disease associated with increased levels of circulating succinate in a patient is selected from the group consisting of obesity, cardiovascular disease, hypertension, type 2 diabetes mellitus, chronic heart failure, ischemic heart disease, ischemia/reperfusion injury and diabetic nephropathy.

38. The method according to claim 34, wherein the ratio of succinate-producing bacteria to succinate-consuming bacteria is the (Prevotellaceae+Veillonellaceae)/(Odoribacteriaceae+Clostridiaceae) ratio.

39. The method according to claim 34, wherein the pharmacological product specifically targets succinate producing bacteria, and wherein the pharmacological product is selected from the group consisting of an antibiotic, an antibacterial antibody, and a bacteriophage.

40. The method according to claim 34, wherein the probiotic product comprises succinate consuming bacteria.

41. The method according to claim 40, wherein the succinate consuming bacteria is selected from the group consisting of Odoribacter spp, Clostridium spp, Phascolarctobacterium spp, Ruminococcus spp, and combinations thereof.

42. The method according to claim 41, wherein the succinate consuming bacteria is selected from the group consisting of Phascolarctobacterium succinatutens, Phascolarctobacterium faecium, Ruminococcus bromii, and Odoribacter laneus.

43. A probiotic product comprising an effective amount of succinate consuming bacteria, wherein the succinate consuming bacteria is selected from the group consisting of Odoribacter spp, Phascolarctobacterium spp, Ruminococcus spp, and combinations thereof.

44. The probiotic product according to claim 43, wherein the succinate consuming bacteria is selected from the group consisting of Phascolarctobacterium succinatutens, Phascolarctobacterium faecium, Ruminococcus bromii, and Odoribacter laneus.
